(19) United States
(12) Patent Application Publication
(10) Pub. No.: US 2021/0407658 A1
(43) Pub. Date: Dec. 30, 2021

(54) DATA PROCESSING SYSTEMS FOR SCHEDULING WORK SHIFTS, SUCH AS PHYSICIAN WORK SHIFTS

(71) Applicant: B9 Systems, LLC, Milton, GA (US)

(72) Inventors: Soha Peter Taheri, Milton, GA (US); Tina Taheri-Khadem, Milton, GA (US)

(21) Appl. No.: 16/915,012

(22) Filed: Jun. 29, 2020

(51) Int. Cl. G16H 40/20; G06N 3/08

(52) U.S. Cl. CPC G16H 40/20; G06N 3/08

(57) ABSTRACT

Computer-implemented data processing methods for electronically scheduling workers to unassigned shifts based on various criteria associated with the unassigned shifts. The system receives from a first business computer, a first set of scheduling data for unassigned work shifts that are available during a scheduling period, identifies one or more users that meet particular criteria associated with each one of the unassigned work shifts, provides notification to a first that a first plurality of the unassigned work shifts are available for assignment, displays the first plurality of unassigned work shifts to the first user, receives a first request to have one or more of the first plurality of unassigned work shifts assigned to the first user, and facilitates the assignment of at least one of the one or more of the first plurality of unassigned work shifts to the first user.

### BACKGROUND

Currently, when staffing work shifts at hospitals, hospital administrators typically: (1) develop a schedule that includes the various work shifts (e.g., shifts of a predetermined number of hours) that need to be filled by physicians, advanced practitioners, nurses, and/or other personnel within an upcoming period of time (e.g., 3 or 6 months); (2) schedule available hospital staff employees to work as many of the work shifts as possible; and then (3) contact qualified independent contractors, or any other suitable individuals (including full or part-time employees of the hospital, who may wish to schedule extra shifts for additional compensation on their off days) to fill any remaining, unfilled work shifts.

The last step referenced above—the process of contacting independent contractors and other suitable individuals and scheduling them to fill the remaining work shifts—is typically labor intensive and time consuming, and often results in poor overall staffing arrangements. As an example, a hospital secretary or other administrator may be given the task of calling independent-contractor physicians, advanced practitioners, and/or nurses to offer them various work shifts, and to update the schedule as appropriate. In doing so, the administrator may spend significant amounts of time trying to reach suitable independent contractors and may not select the best qualified independent contractors to fill the available work shifts. There is currently a need for technology to improve this process.

### SUMMARY

A computer-implemented data processing method for electronically scheduling workers to unassigned shifts based on various criteria of available workers, the method comprising (a) electronically receiving, by one or more computer processors, a first set of scheduling data associated with a first hospital comprising unassigned work shifts that are available during a scheduling period, (b) identifying, by one or more computer processors, one or more users that meet particular criteria associated with each one of the unassigned work shifts in the first set of scheduling data, (c) providing notification to a first user of the one or more users, by one or more computer processors, that a first plurality of the unassigned work shifts are available for assignment, (d) at least partially in response to receiving a request from the first user to view the first plurality of unassigned work shifts, displaying, by one or more computer processors, the first plurality of unassigned work shifts to the first user, (e) receiving from the first user, by one or more computer processors, a first request to have one or more of the first plurality of unassigned work shifts assigned to the first user, and (f) in response to receiving the request, facilitating assignment of at least one of the one or more of the first plurality of unassigned work shifts to the first user. In various embodiments, the method further comprising displaying to the first user, by one or more computer processors, an indication of compensation that the first user will receive if the first user completes one or more of the first plurality of unassigned work shifts. In some embodiments, the step of identifying one or more users that meet particular criteria associated with each one of the unassigned work shifts in the first set of scheduling data further comprises (a) determining, by one or more computer processors, the qualifications of each user, (b) comparing, by one or more computer processors, the qualifications of each user to the minimum qualifications required for each one of the unassigned work shifts in the first set of scheduling data, and (c) selecting, by one or more computer processors, one or more users to notify about the first plurality of the unassigned work shifts. In various embodiments, the step of providing notification to a first user that a first plurality of the unassigned work shifts are available for assignment further comprises determining, by one or more computer processors, whether to provide notification to the first user based, at least in part, on one or more work performance ratings for the first user. In other embodiments, the step of providing notification to a first user that a first plurality of the unassigned work shifts are available for assignment is based on one or more characteristics associated with the first user selected from a group consisting of (a) the first user's work experience, (b) the first user's licensure, (c) ratings of the first user by their peers, (d) ratings of the first user by prior employers, (e) ratings of the first user by patients of the first user, (f) the first user's employment status, and (g) the first user's expected compensation.

In still other embodiments, the method further comprising the steps of (1) electronically receiving, by one or more computer processors, a second set of scheduling data associated with a second hospital comprising unassigned work shifts, (2) identifying, by one or more computer processors, one or more users that meet particular criteria associated with each one of the unassigned work shifts in the second set of scheduling data, (3) providing notification to the first user, by one or more computer processors, that a second plurality of the unassigned work shifts from the second set of scheduling data are available for assignment, (4) at least partially in response to receiving a request from the first user to view the second plurality of unassigned work shifts, displaying, by one or more computer processors, the second plurality of unassigned work shifts to the first user, (5) receiving from the first user, by one or more computer processors, a request to have one or more of the second plurality of unassigned work shifts assigned to the first user, and (6) in response to receiving the second request, facilitating assignment of at least one or the one or more of the second plurality of unassigned work shifts to the first user. In some of these embodiments, displaying the first and second plurality of unassigned work shifts to the first user occurs substantially simultaneously. In other embodiments, the first request and the second request are the same request.

In another embodiment of a computer-implemented data processing method for scheduling workers to unassigned work shifts, the method comprises (a) electronically receiving, by one or more computer processors, a first set of scheduling data associated with a first business indicating a first plurality of unassigned work shifts and at least one criterion for each unassigned work shift, (b) displaying to a first user, by one or more computer processors, a first plurality of unassigned work shifts that are available to the first user, (c) receiving from the first user, by one or more computer processors, a first request to have one or more of the first plurality of unassigned work shifts assigned to the first user, and (d) in response to receiving the first request, facilitating the assignment of the one or more of the first plurality of unassigned work shifts to the user. In various embodiments, the method further comprising (1) electronically receiving, by one or more processors, a second set of scheduling data associated with a second business indicating a second plurality of unassigned work shifts, (2) displaying to the first user, by one or more processors, a second indication that the second plurality of unassigned work shifts are available for assignment, (3) receiving from the first user, by one or more processors, a second request to have one or more of the second plurality of unassigned work shifts assigned to the first user, and (4) in response to receiving the second request, facilitating the assignment of the one or more of the second plurality of unassigned work shifts to the first user. In some embodiments, the method further comprises executing the step of displaying the first indication and the step of displaying the second indication simultaneously, and the first request and the second request are the same request. In other embodiments, the method further comprises determining whether to display the first set of scheduling data to the first user based, at least in part, on one or more work performance ratings for the first user. In various embodiments, the method further comprises determining whether to display the second set of scheduling data to the user based, at least in part, on one or more work performance ratings for the first user. In still other embodiments, the method further comprises displaying to the first user, by one or more processors, an indication of specific compensation that the first user will receive if the user completes one or more of the unassigned work shifts. In some of these embodiments, the method further comprises determining, by one or more processors, the specific compensation based, at least in part, on one or more work performance ratings for the first user. In yet other embodiments, the method further comprises (1) determining, by one or more processors, whether the first user is qualified to work the one or more work shifts, and (2) executing the displaying step at least partially in response to determining that the first user is qualified to work the one or more work shifts.

In yet another embodiments of a computer-implemented data processing method for scheduling work shifts, the method comprises (a) receiving, by one or more computer processors, from a first business computer, a first set of scheduling data comprising first unassigned work shifts for the first business, (b) providing notification to a first user, by one or more computer processors, that a first plurality of the first unassigned work shifts are available for assignment, (c) receiving from the first user, by one or more computer processors, a request to have one or more of the first plurality of first unassigned work shifts assigned to the first user, wherein the request comprises a compensation amount that the first user is willing to accept for each of the one or more of the first plurality of first unassigned work shifts, and (d) in response to receiving the request, facilitating assignment of the one or more of the first plurality of first unassigned work shifts at least partially based on the requested compensation amount. In some embodiments, the step of facilitating assignment of the one or more of the first plurality of first unassigned work shifts is further based on a rating associated with the first user. In other embodiments, the step of providing notification to a first user that a first plurality of the unassigned work shifts are available for assignment further comprises displaying, by one or more computer processors, the first plurality of first unassigned work shifts in a calendar display. In some of these embodiments, the calendar display is integrated with the first user's electronic calendar. In other embodiments, the method further comprises (1) receiving, by one or more computer processors, from a second business computer, a second set of scheduling data comprising second unassigned work shifts for the second business, (b) providing notification to the first user, by one or more computer processors, that a second plurality of the second unassigned work shifts are available for assignment, (c) receiving from the first user, by one or more computer processors, a second request to have one or more of the second plurality of second unassigned work shifts assigned to the first user, wherein the request comprises a compensation amount for each of the one or more of the second plurality of second unassigned work shifts, and (d) in response to receiving the second request, facilitating assignment of the one or more of the second plurality of second unassigned work shifts at least partially based on the requested compensation amount.

A computer-implemented data processing method for electronically offering unassigned shifts to healthcare workers based on various criteria associated with the unassigned shifts and various criteria associated with the healthcare workers, according to various embodiments, may include: (1) obtaining, by one or more processors from one or more data repositories, shift criteria associated with a particular unfilled work shift at a healthcare facility; (2) determining, by one or more processors based on healthcare worker data stored at one or more data repositories, a set of one or more candidates, wherein each of the one or more candidates is associated with a healthcare worker qualified to perform the particular unfilled work shift; (3) determining, by one or more processors, a plurality of pieces of personal information for each respective candidate in the set of one or more candidates; (4) determining, by one or more processors, a respective score for each piece of personal information of the plurality of pieces of personal information; (4) determining, by one or more processors, a plurality of pieces of personnel interaction information for each respective candidate in the set of one or more candidates; (5) determining, by one or more processors, a respective score for each piece of personnel interaction information of the plurality of pieces of personnel interaction information; (6) determining, by one or more processors, a plurality of pieces of shift history information for each respective candidate in the set of one or more candidates; (7) determining, by one or more processors, a respective score for each piece of shift history information of the plurality of pieces of shift history information; (8) determining, by one or more processors based on the scores for each piece of personal information, the scores for each piece of personnel interaction information, and the scores for each piece of shift history information for each respective candidate in the set of one or more candidates, an overall shift acceptance likelihood score for each respective candidate in the set of one or more candidates; ranking, by one or more processors, the set of one or more candidates based on the respective overall shift acceptance likelihood scores for each candidate in the set of one or more candidates; (9) determining, by one or more processors, a subset of highest ranked candidates of the set of one or more candidates; and (10) presenting, by one or more processors on a graphical user interface, an offer to work the particular unfilled work shift to each candidate in the subset of highest ranked candidates of the set of one or more candidates.

In particular embodiments, the method may include: (1) determining that no acceptance of the offer to work the particular unfilled work shift has been received; (2) at least partially in response to determining that no acceptance of the offer to work the particular unfilled work shift has been received, determining a subset of next-highest ranked candidates of the set of one or more candidates, the subset of next-highest ranked candidates of the set of one or more candidates each having a respective overall shift acceptance likelihood score less than any overall shift acceptance likelihood score of any candidate in the subset of highest ranked candidates of the set of one or more candidates; and (3) presenting, on a graphical user interface, a second offer to work the particular unfilled work shift to each candidate in the subset of next-highest ranked candidates of the set of one or more candidates. In particular embodiments, the method may include: (1) determining that no acceptance of the second offer to work the particular unfilled work shift has been received; (2) at least partially in response to determining that no acceptance of the offer to work the particular unfilled work shift has been received, determining that there are no candidates remaining in the set of one or more candidates that have not been presented with one of the offer to work the particular unfilled work shift or the second the second offer to work the particular unfilled work shift; and (3) at least partially in response to determining that there are no candidates remaining in the set of one or more candidates that have not been presented with one of the offer to work the particular unfilled work shift or the second the second offer to work the particular unfilled work shift, presenting an indication to a user that the particular unfilled work shift remains unfilled. In particular embodiments, the method may include: (1) receiving an acceptance of the second offer to work the particular unfilled work shift; and (2) at least partially in response to receiving the acceptance of the second offer to work the particular unfilled work shift, presenting an indication to a user that the particular unfilled work shift is filled. In particular embodiments, the plurality of pieces of personal information for each respective candidate in the set of one or more candidates comprise one or more pieces of information selected from the group consisting of: (a) a home location of the respective candidate; (b) a normal work location of the respective candidate; (c) one or more days of the week on which the respective candidate is available to work; and (d) one or more sets of hours during which the respective candidate is available to work. In particular embodiments, the plurality of pieces of personnel interaction information for each respective candidate in the set of one or more candidates comprise one or more pieces of information selected from the group consisting of: (a) one or more coworkers with whom the respective candidate has worked in the past; (b) one or more peer ratings of the respective candidate; (c) one or more supervisor ratings of the respective candidate; and (d) one or more contacts in a contact list associated with the respective candidate that are scheduled to work the particular unfilled work shift. In particular embodiments, the plurality of pieces of shift history information for each respective candidate in the set of one or more candidates comprise one or more pieces of information selected from the group consisting of: (a) a percentage of acceptances of past offers to fill unfilled work shifts by the respective candidate; (b) an average response time to past offers to fill unfilled work shifts; (c) whether the respective candidate has accepted a past offer to fill an unfilled work shift that occurred in a same time period as the particular unfilled work shift; and (d) whether the respective candidate has accepted a past offer to fill an unfilled work shift that occurred on a same day as the particular unfilled work shift. In particular embodiments, determining the respective score for each piece of personal information of the plurality of pieces of personal information comprises applying a weighting factor to at least one of the plurality of pieces of personal information. In particular embodiments, determining the respective score for each piece of personnel interaction information of the plurality of pieces of personnel interaction information comprises applying a weighting factor to at least one of the plurality of pieces of personnel interaction information. In particular embodiments, determining the respective score for each piece of shift history information of the plurality of pieces of shift history information comprises applying a weighting factor to at least one of the plurality of pieces of shift history information.

A computer system for generating offers to fill unassigned shifts at a healthcare facility, according to various embodiments, may include: at least one computer processor; and computer memory storing computer-executable instructions that, when executed by the at least one computer processor, are operable for: (1) generating, by the at least one computer processor, an ordered list of candidate healthcare workers by: (a) determining one or more scores for one or more respective pieces of personal information for each candidate in the ordered list of candidate healthcare workers; (b) determining one or more scores for one or more respective pieces of personnel interaction information for each candidate in the ordered list of candidate healthcare workers; (c) determining one or more scores for one or more respective pieces of shift history information for each candidate in the ordered list of candidate healthcare workers; (d) determining a respective overall score for each candidate in the ordered list of candidate healthcare workers based on the one or more scores for the one or more respective pieces of personal information, the one or more scores for the one or more respective pieces of personnel interaction information, and the one or more scores for the one or more respective pieces of shift history information for each candidate in the ordered list of candidate healthcare workers; and (e) ordering the candidates in the ordered list of candidate healthcare workers based on the respective overall score for each candidate in the ordered list of candidate healthcare workers; and iteratively presenting, by the at least one computer processor, an offer to work a particular unfilled work shift to each candidate in the ordered list of candidate healthcare workers by: (a) presenting the offer to work the particular unfilled work shift to a first subset of candidates in the ordered list of candidate healthcare workers, wherein each candidate in the first subset of candidates has an overall score greater than a first threshold value; and (b) at least partially in response to determining that no acceptance of the offer to work the particular unfilled work shift has been received, presenting the offer to work the particular unfilled work shift to a second subset of candidates in the ordered list of candidate healthcare workers, wherein each candidate in the second subset of candidates has an overall score less than the first threshold value and greater than a second threshold value.

In particular embodiments, determining the respective overall score for each candidate in the ordered list of candidate healthcare workers based on the one or more scores for the one or more respective pieces of personal information, the one or more scores for the one or more respective pieces of personnel interaction information, and the one or more scores for the one or more respective pieces of shift history information for each candidate in the ordered list of candidate healthcare workers comprises: (1) applying a weighting factor to one or more of the one or more scores for one or more respective pieces of personal information for each candidate in the ordered list of candidate healthcare workers; (2) applying a weighting factor to one or more of the one or more scores for one or more respective pieces of personnel interaction information for each candidate in the ordered list of candidate healthcare workers; and (3) applying a weighting factor to one or more of the one or more scores for one or more respective pieces of shift history information for each candidate in the ordered list of candidate healthcare workers. In particular embodiments, the one or more respective pieces of personal information for each candidate in the ordered list of candidate healthcare workers comprise one or more pieces of information selected from the group consisting of: (a) whether the respective candidate has access to transportation; (b) a distance between a home location of the respective candidate and a location of the particular unfilled work shift; (c) a distance between a normal work location of the respective candidate and the location of the particular unfilled work shift; and (d) an acceptable distance from the home location of the respective candidate that the respective candidate is willing to work. In particular embodiments, the one or more respective pieces of personnel interaction information for each candidate in the ordered list of candidate healthcare workers comprise one or more pieces of information selected from the group consisting of: (a) a frequency with which the respective candidate has worked with one or more coworkers scheduled to work the particular unfilled work shift; (b) a frequency with which the respective candidate has communicated with one or more coworkers scheduled to work the particular unfilled work shift; (c) a request by the respective candidate to not work with one or more coworkers scheduled to work the particular unfilled work shift; and (d) a request by the respective candidate to work with one or more coworkers scheduled to work the particular unfilled work shift. In particular embodiments, the one or more respective pieces of shift history information for each candidate in the ordered list of candidate healthcare workers comprise one or more pieces of information selected from the group consisting of: (a) whether the particular unfilled work shift occurs immediately before a normally scheduled work shift for the respective candidate; (b) whether the particular unfilled work shift occurs immediately after a normally scheduled work shift for the respective candidate; (c) whether the respective candidate has a pending request to swap a scheduled work shift; and (d) whether the respective candidate has ever accepted a past offer to fill an unfilled work shift that occurred on a same day as the particular unfilled work shift.

A non-transitory computer-readable medium storing computer-executable instructions for generating offers to fill unassigned shifts at a healthcare facility, according to various embodiments, may include computer-executable instructions comprising instructions for: (1) determining one or more weighted scores for one or more respective pieces of personal information for each candidate in a plurality of healthcare worker candidates; (2) determining one or more weighted scores for one or more respective pieces of personnel interaction information for each candidate in the plurality of healthcare worker candidates; (3) determining one or more weighted scores for one or more respective pieces of shift history information for each candidate in the plurality of healthcare worker candidates; (4) determining a respective overall score for each candidate in the plurality of healthcare worker candidates by determining a sum of the one or more weighted scores for the one or more respective pieces of personal information, the one or more weighted scores for the one or more respective pieces of personnel interaction information, and the one or more weighted scores for the one or more respective pieces of shift history information for each candidate in the plurality of healthcare worker candidates; (5) generating a ranked listing of the plurality of healthcare worker candidates by ordering the candidates in the plurality of healthcare worker candidates in a descending order based on the respective overall score for each candidate in the plurality of healthcare worker candidates; (6) presenting an offer to work a particular unfilled work shift to each candidate in a first subset of the plurality of healthcare worker candidates, wherein each candidate in the first subset of the plurality of healthcare worker candidates has an overall score within a first predefined percentage of overall score values; and (7) at least partially in response to determining that no acceptance of the offer to work the particular unfilled work shift has been received, presenting the offer to work the particular unfilled work shift to a second subset of the plurality of healthcare worker candidates, wherein each candidate in the second subset of candidates has an overall within a second predefined percentage of overall score values, and wherein the second predefined percentage of overall score values is lower than the first predefined percentage of overall score values.

In particular embodiments, the offer to work the particular unfilled work shift comprises an offer to work a plurality of recurring work shifts that repetitively occur on a same day of the week for a predetermined number of weeks. In particular embodiments, the computer-executable instructions further comprise instructions for: (1) receiving an acceptance of the offer to work the particular unfilled work shift; and (2) at least partially in response to receiving the acceptance of the offer to work the particular unfilled work shift, generating an indication of the acceptance on a graphical user interface. In particular embodiments, the computer-executable instructions further comprise instructions for: (1) receiving no acceptance of the offer to work the particular unfilled work shift; (2) at least partially in response to receiving no acceptance of the offer to work the particular unfilled work shift, determining that there are no candidates remaining in the plurality of healthcare worker candidates that have not been presented with the offer to work the particular unfilled work shift; and (3) at least partially in response to determining that there are no candidates remaining in the plurality of healthcare worker candidates that have not been presented with the offer to work the particular unfilled work shift, generating an indication that the particular unfilled work shift remains unfilled on a graphical user interface. In particular embodiments, the computer-executable instructions further comprise instructions for: determining, based on the particular unfilled work shift, healthcare worker qualifications for the particular unfilled work shift; and selecting, from a data repository, the plurality of healthcare worker candidates based on the healthcare worker qualifications for the particular unfilled work shift.

### BRIEF DESCRIPTION OF THE DRAWINGS

Various embodiments of the invention will be described below. In the course of the description, reference will be made to the accompanying drawings, which are not necessarily drawn to scale, and wherein:

FIG. 1 depicts a Scheduling System 100 according to particular embodiments.

FIG. 2 is a schematic diagram of a computer (such as the Central Scheduling Server 110, or one or more remote computing devices 130 that is suitable for use in various embodiments of the Scheduling System shown in FIG. 1.

FIG. 3A is a flow chart of a scheduling module in accordance with various embodiments for use in the system of FIG. 1.

FIG. 3B is a flow chart of another embodiment of a scheduling module for use with the system of FIG. 1.

FIGS. 4A-41 are screen illustrations of a user interface for use by a user of the system of FIG. 1.

FIGS. 5A-5D are screen illustrations of a user interface for the system of FIG. 1 that illustrates the automatic assignment of work assignments to available providers;

FIGS. 6A-6F are screen illustrations of a user interface for the system of FIG. 1 that illustrates a user manually assigning work assignments to available providers.

FIGS. 7A-7J are screen illustrations of a user interface for the system of FIG. 1 that illustrates how a user swaps a work assignment with another user.

FIGS. 8A-8D are screen illustrations of a user interface for the system of FIG. 1 that illustrates how a facility creates a schedule on the system of FIG. 1.

FIG. 9A-9I are screen illustrations of a user interface for the system of FIG. 1 that illustrate how a provider requests work assignments.

FIG. 10 is a flow chart of a shift candidate scoring module in accordance with various embodiments for use in the system of FIG. 1.

FIG. 11 is a flow chart of a shift fulfillment module in accordance with various embodiments for use in the system of FIG. 1.

### DETAILED DESCRIPTION OF VARIOUS EMBODIMENTS

Various embodiments will now be described in greater detail. It should be understood that the invention may be embodied in many different forms and should not be construed as limited to the embodiments set forth herein. Rather, these embodiments are provided so that this disclosure will be thorough and complete, and will fully convey the scope of the invention to those skilled in the art. Like numbers refer to like elements throughout.

Exemplary Technical Platforms

As will be appreciated by one skilled in the relevant field, the present systems may be, for example, embodied as a computer system, a method, or a computer program product. Accordingly, various embodiments may take the form of an entirely hardware embodiment, an entirely software embodiment, or an embodiment combining software and hardware aspects. Furthermore, particular embodiments may take the form of a computer program product stored on a computer-readable storage medium having computer-readable instructions (e.g., software) embodied in the storage medium. Various embodiments may take the form of web-implemented computer software. Any suitable computer-readable storage medium may be utilized including, for example, hard disks, compact disks, DVDs, optical storage devices, and/or magnetic storage devices.

Various embodiments are described below with reference to block diagrams and flowchart illustrations of methods, apparatuses (e.g., systems), and computer program products. It should be understood that each block of the block diagrams and flowchart illustrations, and combinations of blocks in the block diagrams and flowchart illustrations, respectively, can be implemented by a computer executing computer program instructions. These computer program instructions may be loaded onto a general purpose computer, special purpose computer, or other programmable data processing apparatus to produce a machine, such that the instructions which execute on the computer or other programmable data processing apparatus to create means for implementing the functions specified in the flowchart block or blocks.

These computer program instructions may also be stored in a computer-readable memory that can direct a computer or other programmable data processing apparatus to function in a particular manner such that the instructions stored in the computer-readable memory produce an article of manufacture that is configured for implementing the function specified in the flowchart block or blocks. The computer program instructions may also be loaded onto a computer or other programmable data processing apparatus to cause a series of operational steps to be performed on the computer or other programmable apparatus to produce a computer implemented process such that the instructions that execute on the computer or other programmable apparatus provide steps for implementing the functions specified in the flowchart block or blocks.

Accordingly, blocks of the block diagrams and flowchart illustrations support combinations of mechanisms for performing the specified functions, combinations of steps for performing the specified functions, and program instructions for performing the specified functions. It should also be understood that each block of the block diagrams and flowchart illustrations, and combinations of blocks in the block diagrams and flowchart illustrations, can be implemented by special purpose hardware-based computer systems that perform the specified functions or steps, or combinations of special purpose hardware and other hardware executing appropriate computer instructions.

Overview

Data processing computer systems, according to various embodiments, are adapted to facilitate the scheduling of hospital work shifts by healthcare workers, such as independent contractor physicians, advanced practitioners, and nurses. In particular embodiments, the systems may also be used by other types of healthcare workers, such as physicians, advanced practitioners, and nurses that are on staff at a particular hospital or other types of workers. In further particular embodiments, the systems may also be used in any industry or fields where the coordination of people and/or resources is needed.

A particular embodiment comprises one or more web servers that are collectively adapted to: (1) receive, from a first hospital computer, a first set of scheduling data indicating a first plurality of unassigned work shifts; (2) display to a user (such as an independent-contractor physician, independent-contractor advanced practitioner, or independent-contractor nurse), a first indication that the first plurality of unassigned work shifts are available for assignment; (3) receive, from the user, a request to have one or more of the first plurality of unassigned work shifts assigned to the user; and (4) in response to receiving the request, facilitate the assignment of the one or more of the first plurality of unassigned work shifts to the user.

In particular embodiments, the one or more web servers are in communication with the scheduling servers of multiple health care facilities (e.g., via the Internet or other suitable network) and the system is adapted to display (e.g., simultaneously or non-simultaneously) open work shifts for multiple health care facilities (e.g., hospitals, clinics, or other health care facilities) to the user. This may allow the user to schedule work shifts at multiple hospitals using a single software application.

In various embodiments, the system may be adapted to facilitate the rating of various users of the system by other individuals. For example, the system may be adapted to accept input, from one or more of the user's co-workers and/or patients, regarding: (1) the user's proficiency at their job; (2) the quality of the user's “bedside manner”; (3) the user's ability to work well with others; and/or (4) any other criteria the may be used to evaluate a user. The system may retrieve this information via, for example, one or more suitable surveys (e.g., online surveys).

The system may be adapted to aggregate information from multiple respective surveys from multiple respective users into a single rating score for each user and/or calculate multiple scores for an individual, each score being indicative of the individual's performance in a particular area of competence. In various embodiments, certain ratings may be kept confidential (e.g., they may not be shared with the physician), or may only be shared with a small set of individuals. In certain embodiments, some or all of the ratings may be made publicly available.

The system may, in particular embodiments, be adapted to determine and/or display an indication of compensation that a user will receive for working a particular shift at a particular health care facility. The system may calculate the offered compensation based on, for example, one or more compensation calculation algorithms, which may or may not be received from each participating health care facility. In particular embodiments, these compensation calculation algorithms may be used to calculate the offered compensation for each relevant work shift based on: (1) the desirability of the work shift (e.g., overnight work shifts may be compensated more highly than daytime work shifts); (2) one or more performance ratings associated with the user; (3) the user's seniority (e.g., total years of experience at their job, years of experience working at the particular healthcare facility, etc.); (4) the number of qualified users available to fill the position; and/or (5) any other suitable factor.

In various embodiments, the system may be adapted to time users' access to scheduling work shifts in a way that will increase the chances that the best, least-expensive users will sign up for the available shifts. In particular embodiments, the system may, for example, allow the most desirable users (e.g., the highest rated users, the most senior users, and/or the most specialized users) to access available work shifts and to schedule work within the available work shifts in one or more particular healthcare facilities earlier than other, similar users. This may be done, for example, by staggering the signup times for available work shifts. As an example, when scheduling for a particular six-month period, a hospital may allow: (1) on-staff physicians, advanced practitioners, and nurses to sign up for available shifts during a first time period (e.g., Jun. 1-5, 2017); (2) highly rated physicians, advanced practitioners, and nurses (e.g., physicians, advanced practitioners, and nurses that have been ranked, within the system, with a rating above a particular level (e.g., four out of five stars)) to sign up for any remaining available shifts during a second time period (e.g., Jun. 6-10, 2017); and (3) all remaining candidate physicians, advanced practitioners, and nurses to sign up for any remaining available shifts during a third time period (e.g., Jun. 11-30, 2017). Also, or alternatively, in various embodiments, the system may be adapted to allow healthcare facilities to specify that only individuals satisfying certain specified minimum criteria may sign up for particular work shifts (e.g., only individuals with a rating above a particular specified level may sign up for any, or certain, work shifts). This approach may help to: (1) ensure that the best-performing, least-expensive users are staffed, whenever possible; and (2) incentivize strong workplace performance by rewarding strong performance with more and/or better opportunities to work.

In various embodiments, the system may also, or alternatively, be adapted to allow users to bid on particular available work shifts. In such cases, the system may, for example, allow users to openly bid against other users up to a particular deadline in an on-line auction environment, or the system may allow users to submit their bids in a blind auction. The system may then automatically fill the work shifts based on one or specified particular criteria (e.g., the highest-rated and/or least-expensive bidding user will be assigned to each respective available work shift).

In certain embodiments, the system may be adapted to confirm that each user is properly accredited to work in a particular capacity before scheduling the user to work one or more work shifts. The system may do this, for example: (1) initially, when a user signs up for the system; (2) on a periodic basis; or (3) before scheduling any particular work shift. The system may confirm the user's accreditation by, for example, searching one or more suitable databases to confirm that the user has the proper academic accreditation to serve in the capacity needed to be executed in the work shift; that the user has not had any relevant licenses revoked by any professional governing authority; and/or that the user is current with their continuing medical education requirements. In other embodiments, the system may receive and verify this information directly from the relevant user.

### Example System Architecture

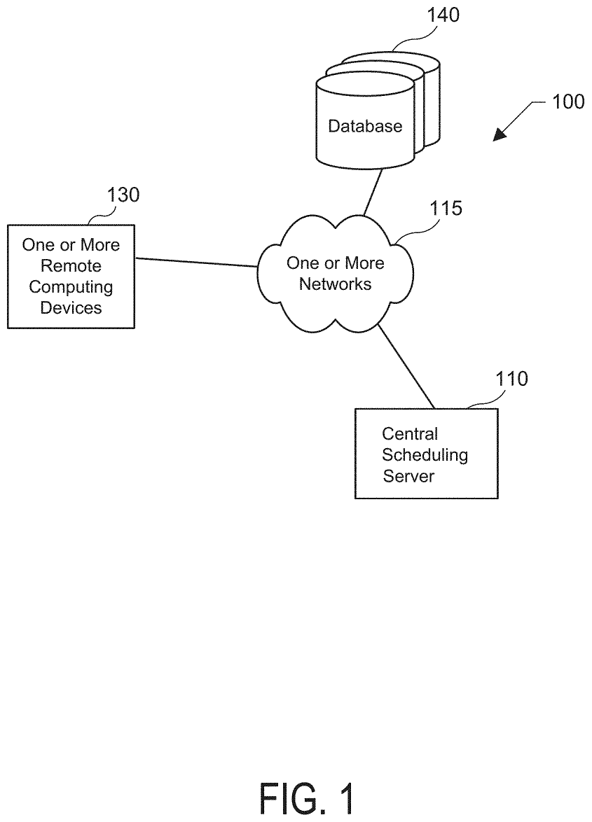

FIG. 1 is a block diagram of a Scheduling System 100 according to a particular embodiment. As may be understood from this figure, the Scheduling System 100 includes Central Scheduling Server 110, one or more networks 115, one or more remote computing devices 130 (e.g., such as a smart phone, a tablet computer, a wearable computing device, a laptop computer, a desktop computer, a remote hospital scheduling server, etc.), and one or more databases 140 (e.g., cloud storage). In particular embodiments, the one or more networks 115 facilitate communication between the one or more remote computing devices 130, the Central Scheduling Server 110 and the one or more databases 140. Any of the one or more databases 140 may be a data repository that captures and/or stores data that may be used to determine potential shift candidates. In addition, or instead, any of the one or more databases 140 may be a data repository configured to capture, store, and/or determine criteria that may be used to determine potential shift candidates.

The one or more networks 115 may include any of a variety of types of wired or wireless computer networks such as the Internet, a private intranet, a mesh network, a public switch telephone network (PSTN), or any other type of network (e.g., a network that uses Bluetooth or near field communications to facilitate communication between computing devices). The communication link between the one or more remote computing devices 130 and the Central Scheduling Server 110 may be, for example, implemented via a Local Area Network (LAN) or via the Internet.

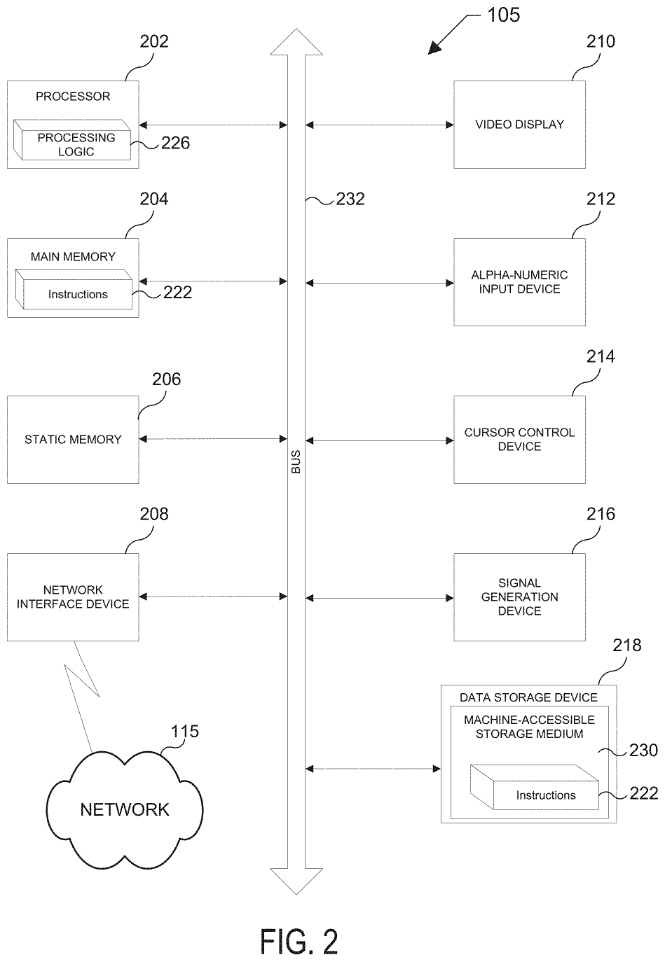

FIG. 2 illustrates a diagrammatic representation of a computer architecture 105 that can be used within the Scheduling System 100, for example, as a client computer (e.g., one of the one or more remote computing devices 130 shown in FIG. 1), or as a server computer (e.g., the Central Scheduling Server 110 shown in FIG. 1).

In particular embodiments, the computing device 105 may be connected (e.g., networked) to other computing devices via a LAN, an intranet, an extranet, and/or the Internet. As noted above, the computing device 105 may operate in the capacity of a server and/or a client computing device in a client-server network environment, and/or as a peer computing device in a peer-to-peer (or distributed) network environment. The computing device 105 may be a desktop personal computing device (PC), a tablet PC, a set-top box (STB), a Personal Digital Assistant (PDA), a cellular telephone, a web appliance, a server, a network router, a switch or bridge, or any other computing device capable of executing a set of instructions (sequential or otherwise) that specify actions to be taken by that computing device. Further, while only a single computing device is illustrated, the term “computing device” shall also be taken to include any collection of computing devices that individually or jointly execute a set (or multiple sets) of instructions to perform any one or more of the methodologies discussed herein.

Returning to FIG. 2, an exemplary computing device 105 includes a processing device 202, a main memory 204 (e.g., read-only memory (ROM), flash memory, dynamic random access memory (DRAM) such as synchronous DRAM (SDRAM) or Rambus DRAM (RDRAM), etc.), a static memory 206 (e.g., flash memory, static random access memory (SRAM), etc.), and a data storage device 218, which communicate with each other via a bus 232.

The processing device 202 represents one or more general-purpose or specific processing devices such as a microprocessor, a central processing unit, or the like. More particularly, the processing device 202 may be a complex instruction set computing (CISC) microprocessor, reduced instruction set computing (RISC) microprocessor, very long instruction word (VLIW) microprocessor, or processor implementing other instruction sets, or processors implementing a combination of instruction sets. The processing device 202 may also be one or more special-purpose processing devices such as an application specific integrated circuit (ASIC), a field programmable gate array (FPGA), a digital signal processor (DSP), network processor, or the like. The processing device 202 may be configured to execute processing logic 226 for performing various operations and steps discussed herein.

The computing device 105 may further include a network interface device 208. The computing device 105 also may include a video display unit 210 (e.g., a liquid crystal display (LCD), a light-emitting diode (LED) display, an organic LED (OLED) display, a cathode ray tube (CRT), etc.), an alphanumeric input device 212 (e.g., a keyboard), a cursor control device 214 (e.g., a mouse), and a signal generation device 216 (e.g., a speaker).

The data storage device 218 may include a non-transitory computing device-accessible storage medium 230 (also known as a non-transitory computing device-readable storage medium or a non-transitory computer-readable medium) on which is stored one or more sets of instructions (e.g., software 222) embodying any one or more of the methodologies or functions described herein. The software 222 may also reside, completely or at least partially, within the main memory 204 and/or within the processing device 202 during execution thereof by the computing device 120—the main memory 204 and the processing device 202 also constituting computing device-accessible storage media. The software 222 may further be transmitted or received over a network 115 via a network interface device 208.

While the computing device-accessible storage medium 230 is shown in an exemplary embodiment to be a single medium, the terms “computing device-accessible storage medium” and “computer-readable medium” should be understood to include a single medium or multiple media (e.g., a centralized or distributed database, and/or associated caches and servers) that store the one or more sets of instructions. The terms “computing device-accessible storage medium” and “computer-readable medium” should also be understood to include any device that is capable of storing, encoding or carrying a set of instructions for execution by the computing device and that cause the computing device to perform any one or more of the methodologies of the present invention. The terms “computing device-accessible storage medium” and “computer-readable medium” should accordingly be understood to include, but not be limited to, solid-state memories, optical and magnetic media, etc.

### Big Data Processing (e.g., Using Neural Networks) to Perform Shift Fulfillment

In various embodiments, the system uses big data techniques to process shift data and candidate data that may have been previously stored to determine one or more candidates to whom to offer particular shifts. The system calculates scores and rankings of various candidates based, at least in part, on the candidate criteria described herein and offers the shifts to candidates based on the scores and rankings. In various embodiments, the system may use big data processing techniques to mine the candidate data previously stored to determine whether to adjust scores and rankings for one or more candidates. In various embodiments, the system may use big data processing techniques to perform any of the processing (e.g., execute any of the modules) described herein to assign resources of any type to one or more requestors of such resources based on any of the various criteria, scores, and/or rankings described herein.

In particular embodiments, one or more neural networks may be used to implement any of the big data processing techniques described herein. A neural network, according to various embodiments, comprises: (1) a plurality of nodes that mimic the operation of the human brain; (2) a training mechanism that analyzes supplied information, and (3) a shift fulfillment engine for determining potential shift candidates, criteria associated with such candidates, and/or scores and/or rankings associated with shift candidates and shifts. The neural network may also perform any of the processing (e.g., execute any of the modules) described herein to assign resources of any type to one or more requestors of such resources based on any of the various criteria, scores, and/or rankings described herein. In various embodiments, each of the nodes comprises one or more weighted input connections, at least one transfer function that combines the inputs, and an output connection. In particular embodiments, the neural network is a variational autoencoder (AE) neural network, a denoising AE neural network, or any other suitable neural network.

In various embodiments, for example, where the system employs a neural network, the system infers information (e.g., regarding shifts, candidates, shift attributes, candidate attributes, candidate preferences, etc.) based on known and/or available data (e.g., shift data, candidate data, etc.). In particular embodiments, the system may use a neural network and integrate inference into its training mechanism to infer information that may then be used in determining potential shift candidates, criteria associated with such candidates, and/or scores and/or rankings associated with shift candidates and shifts.

In various embodiments, the system may use predictive analytics to determine shift information and/or candidates. For example, the system may utilize one or more of a variety of statistical techniques in conjunction with “big data” processing, including as described herein. In particular embodiments, the system may employ any one or more of a variety of statistical techniques in conjunction with data mining, predictive modeling, and/or machine learning (e.g., as described herein) to analyze current shift and/or candidate information and/or historical shift and/or candidate information to generate predictions regarding shift candidates and shifts as described herein. In particular embodiments, the system may employ machine learning in implementing the disclosed systems and methods to better determine shift candidates and suitable shifts.

### Scheduling Module

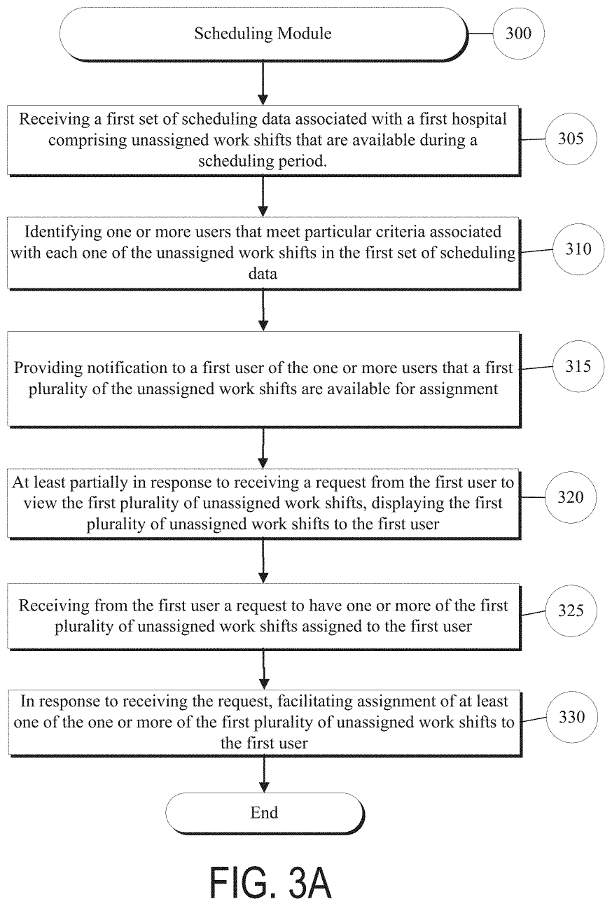

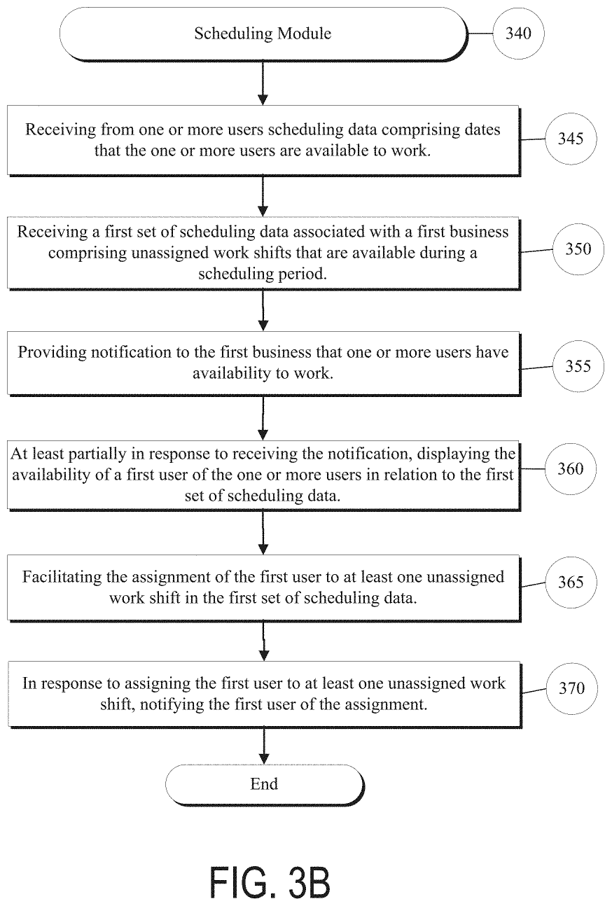

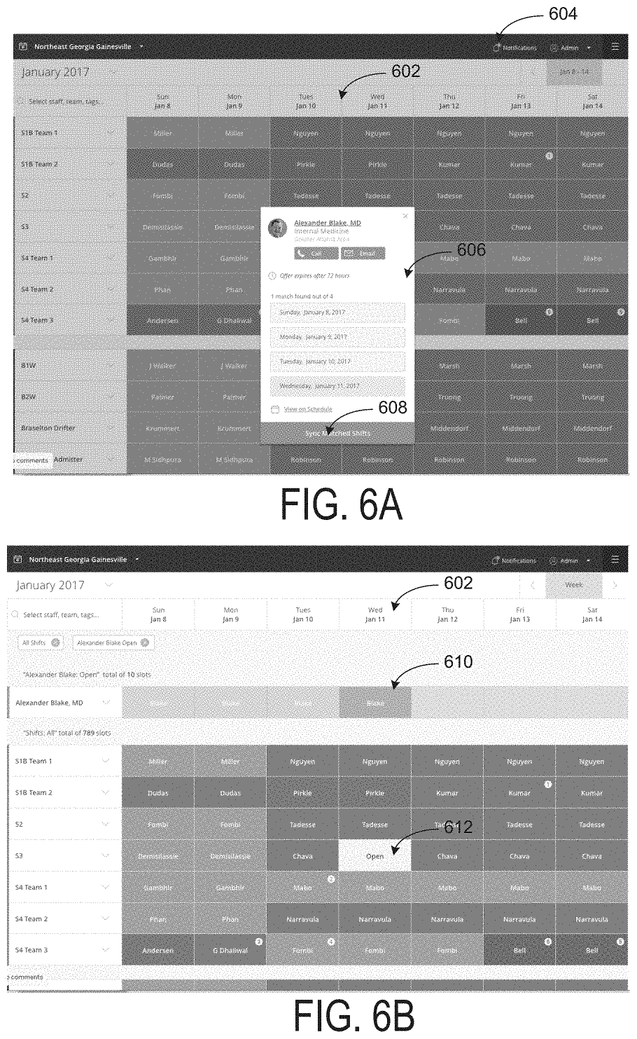

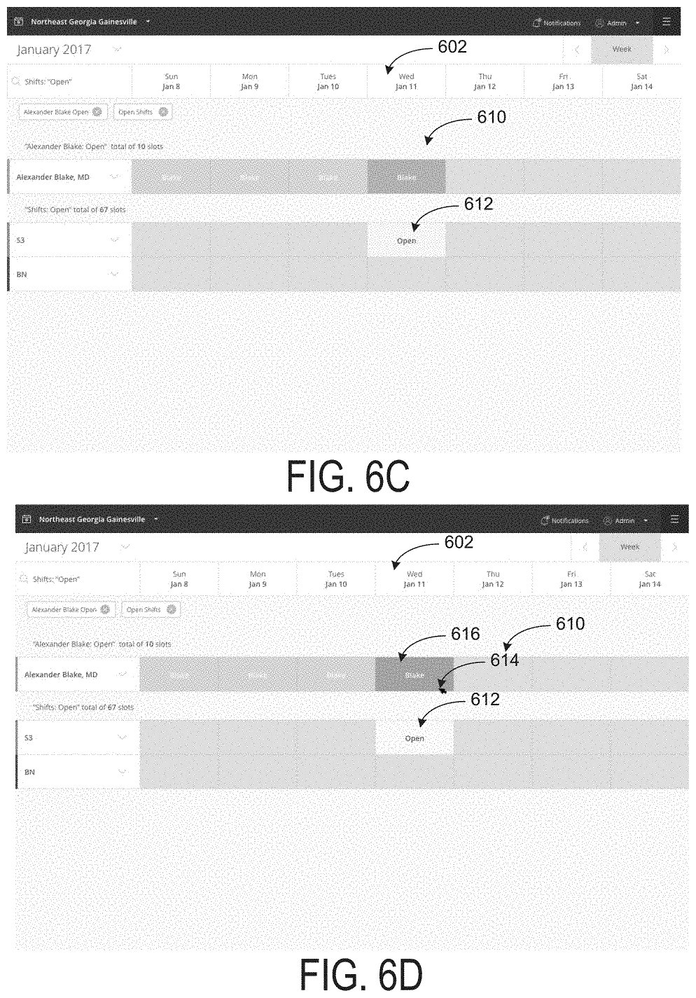

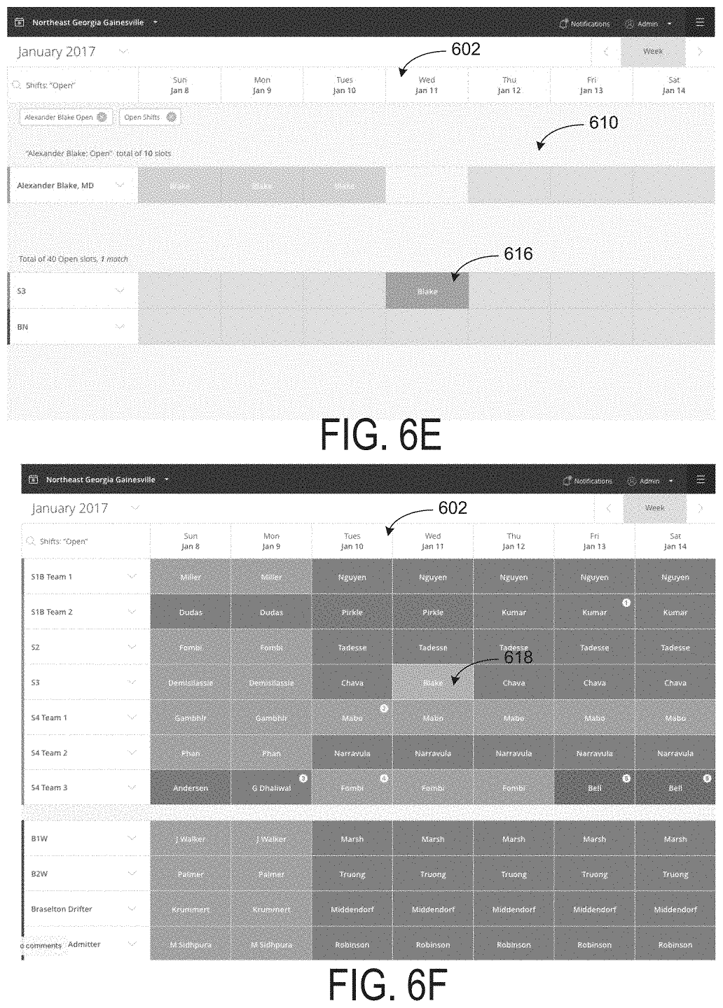

Various aspects of the system's functionality may be executed by certain system modules, including a Scheduling Module 300 (FIG. 3A) that allows users to request certain available shifts and a Scheduling Module 340 (FIG. 3B) that allows users to submit their availability to the system as further described with respect to FIGS. 6A-6F. These modules are discussed in greater detail below. Although these modules are presented as a series of steps, it should be understood in light of this disclosure that various embodiments of the modules described herein may perform the steps described below in an order other than in which it is presented. In still other embodiments, the modules may omit certain steps described below. In various other embodiments, the modules described herein may perform steps in addition to those described in the modules and/or instead of those steps described in the modules. In still other embodiments, the modules described herein may perform some steps from one module and other steps from the other module. In particular embodiments, the system may include a neural network comprising nodes that mimic the operation of the human brain, a training mechanism that analyzes supplied information (e.g., health care worker data, shift data, scheduling data, etc.), and a shift fulfillment engine for determining and ranking candidates for filling one or more shifts, for offering shifts to candidates, and for entering candidate information into a schedule when shifts are accepted by candidates.

### First Embodiment of the Scheduling Module

Electronically Receiving a First Set of Scheduling Data Associated with a First Hospital Comprising Unassigned Work Shifts that are Available During a Scheduling Period.

In particular embodiments, when executing the Scheduling Module 300 shown in FIG. 3A, the system begins, at Step 305, by receiving a first set of scheduling data. The scheduling data may be received from any number of businesses, not for profit organizations, etc. For example, in the case of a hospital, the hospital may have full-time staff that is assigned to 75% of work shifts available at the hospital. The remaining shifts may be filled by contract employees. This enables the hospital to reduce costs by not over staffing. Following the example of a hospital, a first scheduling server, which is associated with a first hospital, and a second scheduling server, which is associated with a second hospital, are connected, via a suitable network (e.g., the Internet or any other suitable network) to a central scheduling server 110, which may, for example, be a central web server. In advance of an upcoming particular scheduling period (e.g., a particular three-month scheduling period), a scheduling representative of the first hospital and a scheduling representative of the second hospital will generate a list of work shifts that need to be filled within that scheduling period. For example, in various embodiments, each hospital may have a certain number of work shifts for various positions that must be filled each day of a scheduling period. Thus, the available work shift information, which may include, the day, time, qualifications to work the position, compensation offered, etc. is transmitted via a data file to the central scheduling server.

Identifying One or More Users that Meet Particular Criteria Associated with Each One of the Unassigned Work Shifts in the First Set of Scheduling Data

Next, at Step 310, the system identifies one or more users of the system that meet particular criteria associated with each one of the unassigned work shifts received in the scheduling data from one or more enterprises (e.g., hospital, business, not-for-profit, etc.). Continuing with the hospital example, the system may have one or more registered users. When a user registers with the system, the user is required to establish an account on the system and enter all of their pertinent information including, but not limited to, name, address, telephone number, work history, education history, certifications and associated license information (e.g., board certification numbers, registered nurse certificate numbers, etc.). In various embodiments, the system upon receiving user registration information then verifies the accuracy of the submitted information. In other embodiments, the information supplied by a user may be verified after a user is registered. Verification may be done automatically by linking to State Boards, national board, certifying entities, etc. In other embodiments, the information may be manually verified. In still other embodiments, the information supplied by a user may be both automatically and manually verified.

In certain embodiments, the system may be adapted to confirm that each user is properly accredited to work in a particular capacity before providing notification to the user or scheduling the user to work one or more work shifts. The system may do this, for example: (1) initially, when a user signs up for the system; (2) on a periodic basis; or (3) before scheduling any particular work shift. The system may confirm the user's accreditation by, for example, searching one or more suitable databases to confirm that the user has the proper academic accreditation to serve in the capacity needed to be executed in the work shift; that the user has not had any relevant licenses revoked by any professional governing authority; and/or that the user is current with their continuing medical education requirements. In other embodiments, the system may receive and verify this information directly from the relevant user.

In particular embodiments, once the user information has been verified, the user's account may be activated so that the user may be selected (e.g. identified by the system) for one or more listed vacancies. In various embodiments, the system may be configured to identify one or more users that meet particular criteria associated with an unassigned work shift when a user meets the minimum certification requirement for the unassigned work shift. For example, when an unassigned work shift is for a shift in an emergency room, the system may identify all boarded doctors since any boarded doctor may work a shift in an emergency room. In another example, for an unassigned work shift in radiology, the system may identify all boarded radiologists as the one or more users.

In some embodiments, the system may identify the one or more users based on one or more criteria. For example, for the emergency room shift, the first criteria may be a boarded doctor and the second criteria may be having a 4 star rating from patients. Thus, only boarded doctors that have at least a 4 star rating would be identified by the system for the unassigned work shift. It should be understood that the system may identify the one or more users based on any one of a number of available criteria. For example, the system may be configured to indemnity one or more users based on the minimum compensation that the user requires to fill an unassigned work shift. Thus, if the compensation for a work shift is below the minimum compensation that a user is willing to accept, the user will not be identified by the system as a potential candidate for the unassigned work shift.

Providing Notification to a First User of the One or More Users that a First Plurality of the Unassigned Work Shifts are Available for Assignment

In various embodiments, the Scheduling System 100, at Step 315, provides notification to a first user of the one or more users that a first plurality of the unassigned work shifts are available for assignment. The first user is selected from the identified one or more users who meet particular criteria associated with each of the unassigned work shifts. In various embodiments, the first plurality of the unassigned work shifts may comprise at least one work shift that the first user qualifies for. In other embodiments, the first plurality of the unassigned work shifts may comprise multiple unassigned work shifts that the first user qualifies for based on criteria set by the business.

In various embodiments, the notification may comprise one or more of sending an e-mail to the first user, sending a text message to the first user, providing a numeric notification on an application icon that one or more unassigned work shifts are available to the first user. It should be understood that there are many other suitable notifications that the system may use such as providing a telephone call to the first user. In still other embodiments, the notification may be provided by showing an indication of the number of available shifts on the icon for the program.

In various embodiments, the system may be adapted to time users' notification and/or access to scheduling work shifts in a way that will increase the chances that the best, least-expensive users will sign up for the available shifts. In particular embodiments, the system may, for example, notify and allow the most desirable users (e.g., the highest rated users, the most senior users, and/or the most specialized users) to access available work shifts and to schedule work within the available work shifts in one or more particular healthcare facilities earlier than other, similar users. This may be done, for example, by staggering the notification times and/or signup times for available work shifts. As an example, when scheduling for a particular six-month period, a hospital may allow: (1) on-staff physicians, advanced practitioners, and nurses to sign up for available shifts during a first time period (e.g., Jun. 1-5, 2017); (2) highly rated physicians, advanced practitioners, and nurses (e.g., physicians, advanced practitioners, and nurses that have been ranked, within the system, with a rating above a particular level (e.g., four out of five stars)) to sign up for any remaining available shifts during a second time period (e.g., Jun. 6-10, 2017); and (3) all remaining candidate physicians, advanced practitioners, and nurses to sign up for any remaining available shifts during a third time period (e.g., Jun. 11-30, 2017).

Also, or alternatively, in various embodiments, the system may be adapted to allow healthcare facilities to specify that only individuals satisfying certain specified minimum criteria may sign up for particular work shifts (e.g., only individuals with a rating above a particular specified level may sign up for any, or certain, work shifts). This approach may help to: (1) ensure that the best-performing, least-expensive users are staffed, whenever possible; and (2) incentivize strong workplace performance by rewarding strong performance with more and/or better opportunities to work.

In various embodiments, the system notification may, for example, include transmitting suitable electronic messages to the user when it is possible for the user to sign up for available shifts. The notification may also include, for example, a link to a secure website that the user may use to log into to view available work shifts that are available to sign up for. The website, which may be supported by the central scheduling server, may display available work shifts in any suitable manner (e.g., in the form of entries on a graphical calendar, as lists, as text, etc.). The work shifts may include work shifts for any suitable number of different health care facilities, which may, for example, allow independent-contractor healthcare workers to easily prepare cohesive, non-conflicting work schedules at multiple healthcare facilities using the system.

At Least Partially in Response to Receiving a Request from the First User to View the First Plurality of Unassigned Work Shifts, Displaying the First Plurality of Unassigned Work Shifts to the First User

In various embodiments, the system at Step 320, displays the first plurality of unassigned work shifts to the first user at least partially in response to receiving a request from the first user to view the unassigned work shifts. In various embodiments, where the notification is via a text or program notification, when the first user selects the text of program notification the program may be configured to open to display the available unassigned work shifts to the first user. In another embodiment, a software program on the first user's handheld device (e.g., tablet, smart phone, etc.) or computer may integrate with the first user's electronic calendar. In these embodiments, the available unassigned work shifts may tentatively show up on the first user's calendar to allow the first user to compare their availability to fill the unassigned work shift with their current schedule. In other embodiments, the program may have its own user interface such as a calendar view that shows the available unassigned work shifts. In some of these embodiments, the program calendar view may integrate with the electronic calendar on the handheld device so that the unassigned work shifts may be shown in reference to other appointments already scheduled by the first user.

In various embodiments, the system is adapted to display (e.g., simultaneously or non-simultaneously) open work shifts for multiple organizations (businesses, business locations, institutions, health care facilities (e.g., hospitals, clinics, or other health care facilities), etc.) to the user. This allows users to schedule work shifts at multiple organizations using a single software application.

The system may, in particular embodiments, be adapted to determine and/or display an indication of compensation that a user will receive for working a particular shift at a particular health care facility. The system may calculate the offered compensation based on, for example, one or more compensation calculation algorithms, which may or may not be received from each participating health care facility. In particular embodiments, these compensation calculation algorithms may be used to calculate the offered compensation for each relevant work shift based on: (1) the desirability of the work shift (e.g., overnight work shifts may be compensated more highly than daytime work shifts); (2) one or more performance ratings associated with the user; (3) the user's seniority (e.g., total years of experience at their job, years of experience working at the particular healthcare facility, etc.); (4) the number of qualified users available to fill the position; and/or (5) any other suitable factor.

Receiving from the First User a Request to have One or More of the First Plurality of Unassigned Work Shifts Assigned to the First User

In various embodiments, the system 100, at Step 325, is configured to receive a request from a user to have one or more of the unassigned work shifts assigned to the user. The request may be made via any suitable mode of communication. For example, in various embodiments, the user may select one or more of the unassigned work shifts that are displayed to the user in Step 320 and send a request to the system that the user wishes to accept those unassigned work shifts. In some embodiments, the user can do this by selected one or more unassigned work shifts from a list of unassigned work shifts that the user qualifies for and select an accept button on the user interface that notifies the system that the user is interested in working the selected shifts. In other embodiments, the available work shifts may be displayed on a calendar display that allows the user to select the days that the user is interested in working. In this way, if there is more than one work shift available on that day, the user may configure account settings to select the best suited work shift that is available on that day. For example, the user setting may allow the system to select one of multiple work shifts available on the selected date based on one or more of (1) the highest paying work shift available on that day, (2) the closest work location to a predefined location (e.g., the user's home address), (3) the highest rated facility, or any other suitable criteria.

In various embodiments, the system 100 may also, or alternatively, be adapted to allow a user to bid on particular available work shifts. In such cases, the system may, for example, allow users to openly bid against other users up to a particular deadline in an on-line auction environment, or the system may allow users to submit their bids in a blind auction. The system may then automatically fill the work shifts based on one or specified particular criteria (e.g., the highest-rated and/or least-expensive bidding user will be assigned to each respective available work shift).

In Response to Receiving the Request, Facilitating Assignment of at Least One of the One or More of the First Plurality of Unassigned Work Shifts to the First User

In various embodiments, the system 100, at Step 330, in response to receiving the request from the user, facilitate assignment of at least one of the one or more first plurality of unassigned work shifts to the first user. The system facilitates assignment to the user based on one or more factors that are set up by the submitter of the unassigned work shifts. For example, in the hospital example, the system may select the assigned user based on user rating, requested compensation, user availability based on other user requested work assignment, etc. Thus, if a user requests multiple work assignment from the available work assignments, the system may award assignments to maximize a particular user's ability to accept multiple available work assignments. Said another way, for a highly rated user, the system may optimize this particular user's requests so that they are assigned most or all of their requested assignments, whereas a lower rated users may only get one of their requested assignments.

### Second Embodiment of the Scheduling Module

In a second embodiment of a Scheduling Module 340, the module begins at Step 345, by electronically receiving scheduling data from one or more users that comprises dates that the one or more users are available to work. The scheduling data may also include additional information about the user such as licensure information, national provider identification number or any other information associated with the user that is necessary to allow the user to be assigned a work shift. Next at Step 350, the system 100 receives a first set of scheduling data associated with a first business (e.g., a hospital, etc.) comprising unassigned work shifts that are available during a scheduling period. For example, in a hospital setting, the unassigned work shifts would correspond to one or more work shifts for medical doctors, nurses, healthcare providers, etc.

At step 355, the system 100 provides notification to the first business that one or more users have availability to work. In various embodiments, the notification may be sent to a hospital administrator, a scheduling clerk, the staff supervisor or any other suitable party that is in charge of scheduling. At Step 360, at least partially in response to receiving the notification, the system 100 displays the availability of a first user of the one or more users in relation to the first set of scheduling data. In various embodiments, the first user data may be shown in a calendar view aligned with the first set of scheduling data, as shown in FIG. 6C.

At Step 365, the system 100 facilitates the assignment of the first user to at least one unassigned work shift in the first set of scheduling data. In various embodiments, the system 100 may automatically assign the first user to one or more unassigned work shifts in the first set of scheduling data based on one or more criteria (e.g., user rating, compensation requested, etc.) associated with the first user. In other embodiments, the system 100 may allow for manual assignment of the first user to one or more of the unassigned work shifts. Finally, at Step 370, the system 100 in response to the assignment notifies the first user of the one or more work assignments.

### Alternative Features for the Scheduling Module

In various embodiments, the system 100 may be adapted to facilitate the rating of various users of the system by other individuals. For example, the system may be adapted to accept input, from one or more of the user's co-workers and/or patients, regarding: (1) the user's proficiency at their job; (2) the quality of the user's “bedside manner”; (3) the user's ability to work well with others; and/or (4) any other criteria the may be used to evaluate a user. The system may retrieve this information via, for example, one or more suitable surveys (e.g., online surveys).

The system may be adapted to aggregate information from multiple respective surveys from multiple respective users into a single rating score for each user and/or calculate multiple scores for an individual, each score being indicative of the individual's performance in a particular area of competence. In various embodiments, certain ratings may be kept confidential (e.g., they may not be shared with the physician), or may only be shared with a small set of individuals. In certain embodiments, some or all of the ratings may be made publicly available.

### Example Operation of the System

The structure and operation of a particular system is described below. It should be understood that this discussion is intended for example purposes only and is not intended to be limiting.

User Registration with the System

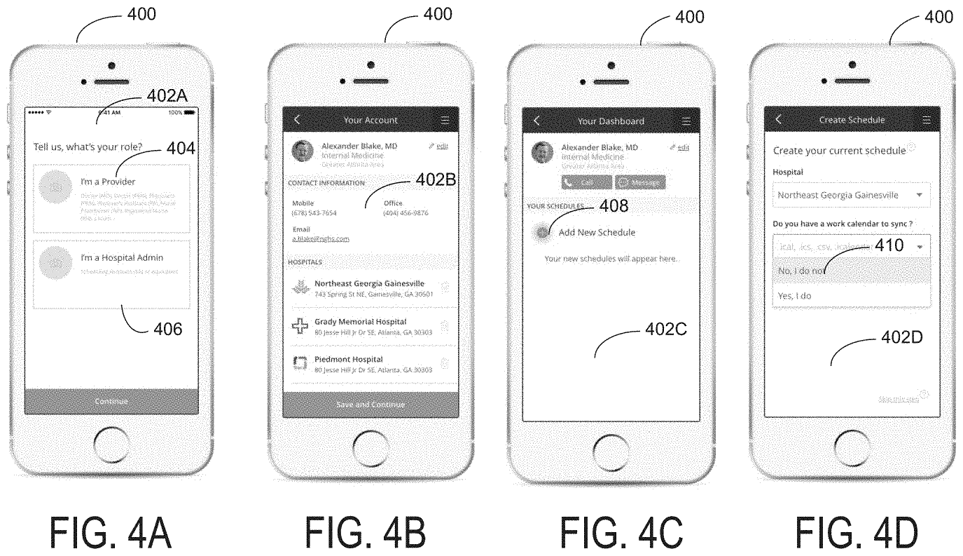

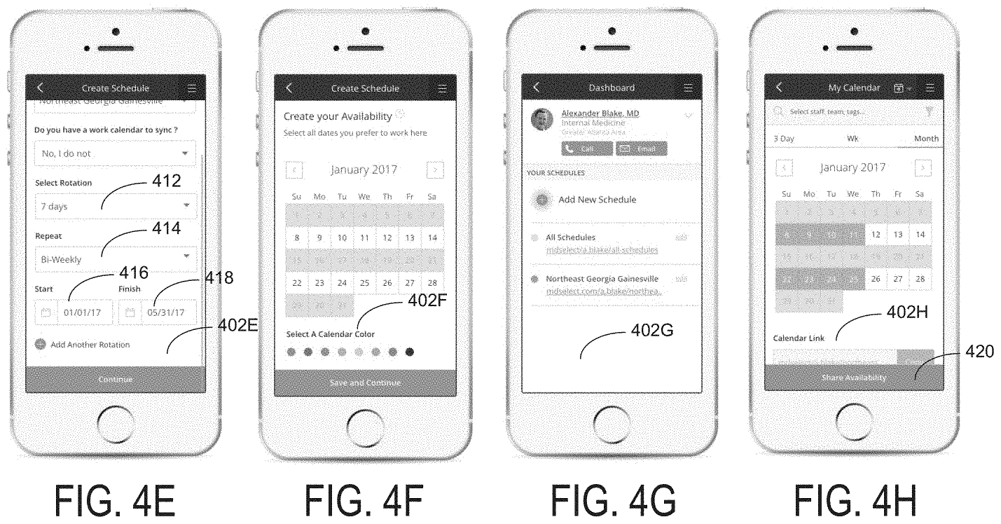

In various embodiments and referring to FIGS. 4A-41, a user begins use of the system by downloading the application to a computing device 400. It should be understood, that in various embodiments, the user may instead register using a web application, or webpage. Once the application is downloaded to the computing device 400, selects to register with the system 100 by entering their name, address, telephone number, e-mail address. Once the basic user information is submitted, the user interface 402A provides the user with a provider item 404 or a business item 406 (e.g., a hospital, etc.) that allows the user to select their role in the system 100. In the present example, the user selects the provider item 404 indicating that they are a provider that is seeking open work shifts. The system 100 then asks the user what hospitals that they work at (e.g., what hospitals that they are credentialed at, etc.) and provides a drop down menu for the user to select from. The user may select one or more hospitals that they are affiliated with. The system may also be configured to obtain licensure information, national provider identification number or any other information that is necessary from the user.

Referring to FIG. 4B, the user interface 402B then displays the user's account information. Referring to FIG. 4C, the user interface 402C next presents the user with the option to add their schedule to their account by selecting an “add new schedule” item 408. Referring to FIG. 4D, the user interface 402D next provides the user with the ability to select whether they wish to sync a work calendar with their account on system 100. In the present example, the user does not have a work calendar so they select the “no, I do not” menu item 410, which brings the user to the user interface 402 shown in FIG. 4E. Referring to FIG. 4E, the user interface 402E allows the user to select their present work rotation at menu item 412, how often the rotation repeats at menu item 414, and a start date 416 and an end date 418 for their current schedule. Next at FIG. 4F, the user interface 402F allows the user to select their current availability to work additional shifts. Once the user completes the process to add their schedule(s), the system displays a list of their current schedules at user interface 402G (FIG. 4G). The user may then share their current availability with the system 100 by selecting a “share availability” menu item 420 on user interface 402H (FIG. 4H). Once the user selects the “share availability” menu item 420, the system presents the user with user interface 402I (FIG. 41) to allow the user to share their availability with multiple businesses (hospitals, etc.) by selecting one or more recipients at menu item 422, an indication of the duration of their availability offer at menu item 424, and a predetermined time frame for advanced notification of a work shift start at menu item 426. Some of the information, such as the duration and the prior notification may be optional information in various embodiments. At this point, the system 100 receives the user availability information.

Automatic and Manual Work Shift Assignments

In particular embodiments, a first scheduling server, which is associated with a first hospital, and a second scheduling server, which is associated with a second hospital, are connected, via a suitable network (e.g., the Internet or any other suitable network) to a central scheduling server, which may, for example, be a central web server. In advance of an upcoming particular scheduling period (e.g., a particular three-month scheduling period), a scheduling representative of a first hospital generates a list of work shifts that need to be filled within that scheduling period. Each work shift identifies the start and stop time of the work shift, the types of healthcare professionals needed to fulfill each work shift (e.g., general surgeon, anesthesiologist, surgical assistant, etc.) and the location where the work is to be performed (e.g., Operating Room 4 at North Florida Regional Hospital in Gainesville, Fla.). A scheduling representative at a second hospital also generates a similar list of work shifts that need to be filled for the upcoming time period at the second hospital.

The respective lists of upcoming work shifts for the first and second hospitals are then transmitted to the central scheduling server via the Internet (e.g., using a suitable application programming interface (API)). The central scheduling server may also receive, from each of the first and second hospitals, an indication of how each of the first and second hospitals wishes to staff the work shifts, how each hospital is willing to compensate available independent health care workers for each work shift and/or type of work shift, etc.

For example, the first hospital and second hospital may both specify that: (1) on-staff physicians, advanced practitioners, and nurses are to be allowed to sign up for available shifts beginning on a first date (e.g., Jun. 1, 2017); (2) highly-rated independent-contractor physicians, advanced practitioners, and nurses (e.g., physicians, advanced practitioners, and nurses that have been ranked, within the system, with a rating above a particular level (e.g., four out of five stars)) are to be allowed to sign up for available shifts beginning on a second date (e.g., Jun. 6, 2017); and (3) all remaining candidate physicians, advanced practitioners, and nurses are to be allowed to sign up for any remaining available shifts beginning on a third date (e.g., Jun. 12, 2017). The hospital may further specify that the signup window for the scheduling period will close on a specific date (e.g., Jun. 30, 2017).

The first hospital may specify a first standard compensation level for each shift, and also specify circumstances under which healthcare workers may be compensated more or less than the standard compensation level for a particular shift. For example, the first hospital may specify that independent contractor healthcare workers who work overnight shifts will be paid 30% more than the standard compensation level for that type of shift. Similarly, the first hospital may specify that independent-contractor healthcare workers who currently have performance ratings above a particular rating level will be paid 15% more than other, similarly qualified independent-contractor heath care workers working the same shift. The second hospital may provide a similar compensation schedule.

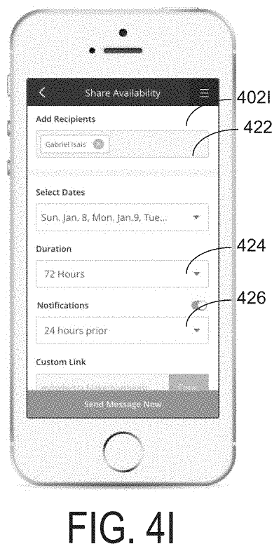

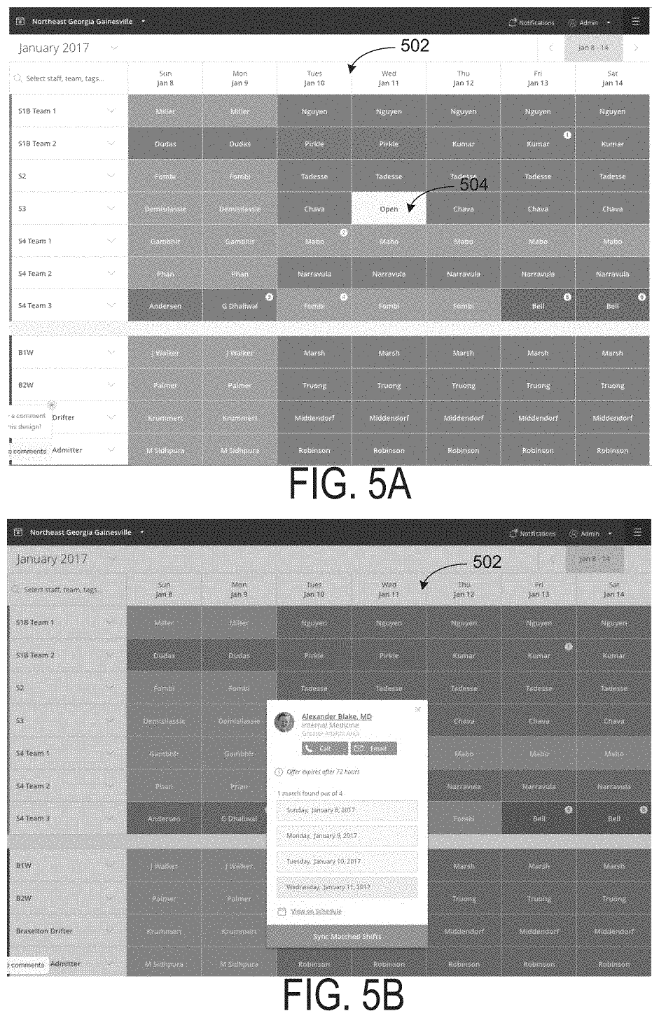

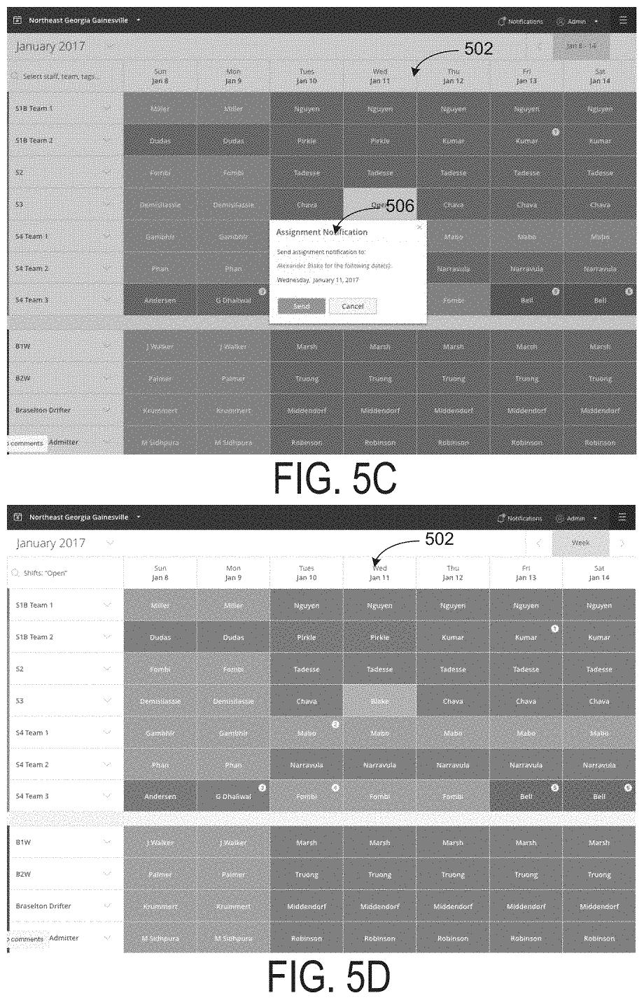

Referring to FIG. 5A, assuming the first hospital account with the system 100 is set so that shifts are automatically scheduled based on one or more of the criteria discussed above (e.g., compensation, rating, etc.), the system 100 populates a calendar 502 of the shifts that have been scheduled and the shifts that are open 504. Open shifts 504 may be shown in a different color to distinguish them from assigned shifts. Referring to FIG. 5B, the system selects user availability that has been transmitted from users and looks for user availability that matches the open shifts 504 on the calendar 502. Referring to FIG. 5C, once the system 100 assigns an open shift 504 to the user, the system 100 send a notification 506 of the assignment. In various embodiments, a separate notification may be sent for each assigned work shift. In other embodiments, a single notification may be sent for all assigned work shifts to the user. Referring to FIG. 5D, once the open work shift has been assigned, the system shows the previously open work assignment as an assigned work assignment and indicates the name of the user assigned that work shift.

Referring to FIG. 6A, if the first business has their account set to manual assignment, then a scheduling assistant may manually assign open work shifts to users. For example, similar to FIG. 5A, a shift calendar 602 is created by the system 100. When the system receives a notification from a user of a request for open shifts, the notification is indicated by the system at 604. When the scheduling assistant selects the notification icon 604, a notification window 606 opens that displays the user's scheduling request. The scheduling assistant can select a “synch matched shifts” menu item 608 that places an availability overlay 610 for the user on the calendar display 602, as shown in FIG. 6B. Referring to FIG. 6B, the user availability in the availability overlay 610 is aligned with the shift calendar 602 to clearly display the user's availability with open shifts 612 shown on the calendar. Referring to FIG. 6C, the scheduling assistant can filter the shift calendar 602 so that it only shows the open shifts instead of both open shifts and assigned shifts. In the filtered view, the scheduling assistant may hover a pointer 614 over the assignment request date that aligns with an open work assignment 612 and drag the selected work box 616 into the open work shift, as shown in FIG. 6E. As a result, when the scheduling assistant switches from the filtered mode into the unfiltered mode shown in FIG. 6F, the calendar 602 shows all work shifts, including the newly assigned work shift 618.

As users sign up for various shifts, the central scheduling server may transmit updated respective scheduling information to each respective hospital scheduling server according to any appropriate schedule (e.g., in real time, nightly, weekly, etc.). For example, the central scheduling server may transmit updated scheduling information for the first hospital to the first hospital in real time so that the first hospital's scheduling server retains an up-to-date schedule of the work shifts. The central scheduling server may transmit updated scheduling information for the second hospital to the second hospital in real time so that the second hospital's scheduling server retains an up-to-date schedule of the work shifts.

At the closing date/time for signup for the particular scheduling time period, the system 100 may optionally generate and transmit a suitable electronic message to any participating users and/or hospitals indicating that the scheduling time period has closed. If any work shifts remain unfilled at that time: (1) the system may open an extended time period that may, for example, include additional incentives for signing up for the unfilled work shifts (e.g., a 10% compensation bonus for those work shifts); or (2) a suitable human representative may work to manually fill those positions (e.g., with staff healthcare workers).

Automated Swapping of Work Shifts Between Users

In various business settings, the swapping of work shifts between employees and contractors is a difficult task and may require the intervention of a third party to facilitate a work shift change such as human resources, a manager, a scheduler or another person. Thus, swapping work shifts becomes an arduous task that cannot be accomplished efficiently and on short notice. The scheduling system 100 described herein provides a quick and efficient method of automating the swapping of work shifts between users without the help of a third party.

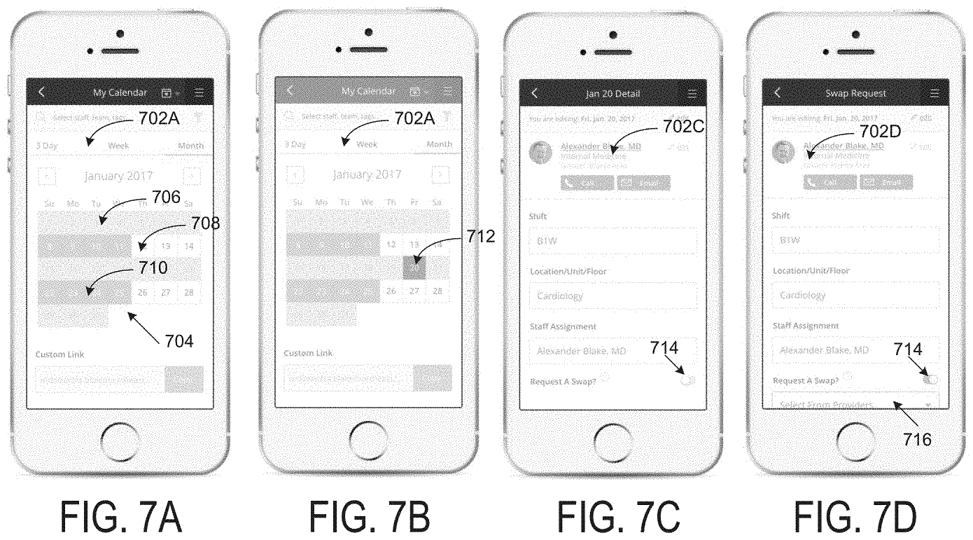

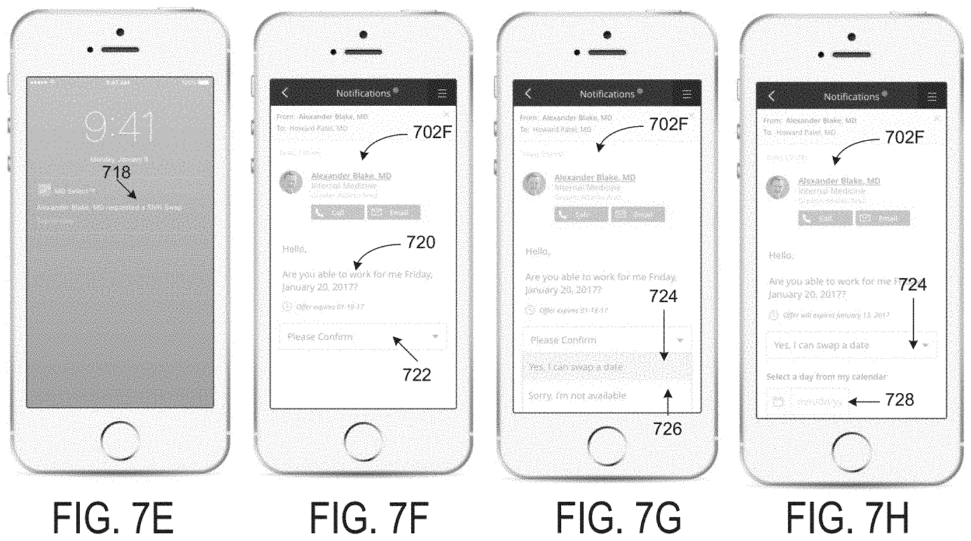

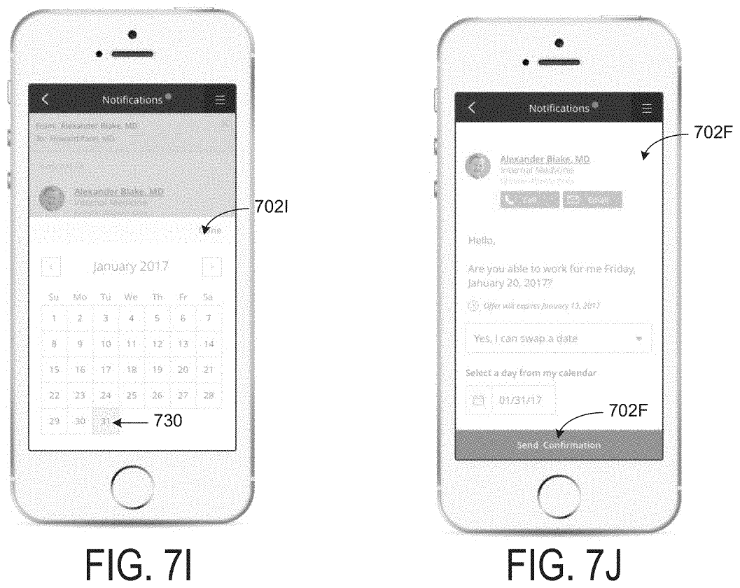

Referring to FIGS. 7A-7J, the system 100 allows a user to easily swap a work assignment with another user. Referring to FIG. 7A, the system presents a first user's work schedule via a user interface 702A. The schedule may be in a calendar form 704 where scheduled work days 706 are shown in gray, open days 708 are shown in white and requested work days 710 are shown in green. Referring to FIG. 7B, if the first user needs to swap a work shift with another user the first user may select the work shift 712, which becomes highlighted. Referring to FIG. 7C, after the work shift is selected, a user interface 702C opens that displays the details of the work shift and allows the first user to select an option 714 to request to swap the work shift. Referring to FIG. 7D, when the first user selects the option 714, a menu item 716 activates that allows the first user to select another provider (e.g., a second user) to which a work shift swap request will be sent. The system 100 may be configured to only list providers that are available to swap with the first user. Said another way, the system 100 may be configured to only list providers that do not have a work shift conflict with the work shift that the first user is trying to swap out. Referring to FIG. 7E, once the first user selects the second user and selects enter, an electronic notification 718 is sent to the selected second user. The electronic notification may be a text message with a link that opens a program, it may be a notification for the program, or it may be any other electronic notification.

Referring to FIG. 7F, when the second user selects the electronic notification 718, the second user is directed to a user interface 702F that provides an indication 720 to the second user that the first user is requesting a work shift swap. The user interface 702F also provides a selection menu item 722 that allows the second user to confirm whether they are willing to swap shifts. Referring to FIG. 7G, when the second user selects the menu item 722, a dropdown box 724 opens and allows the user to select either a “yes, I can swap a date” 726 or a “Sorry, I'm not available” 728. Referring to FIG. 7H, if the second user selects the “yes, I can swap a date” 726 from the drop down menu 722, the user interface 702F presents the second user with a “select a day from my calendar” menu items 728 that allows the second user to select a work shift to swap with the first user. Referring to FIG. 7I, when the user clicks on the “select a day from my calendar” menu items 728 (FIG. 7H), a calendar user interface 7021 opens to allow the user to select a work shift 730 from the calendar user interface 7021. Referring to FIG. 7J, once the second user selects the work shift 730 that they are willing to swap for the work shift that was requested by the first user, the second user can select a “send confirmation” menu item 732 on user interface 702 to send a swap confirmation back to the first user.

At this point, the system 100 updates the schedule to include that the second user is now working the shift on Jan. 20, 2017 and the first user is now scheduled to work the shift on Jan. 31, 2017. The system 100 may also be configured to transmit the shift changes back to the first hospital scheduling server so that the data at the first hospital is up to date. In various embodiments, the system 100 may be configured to allow the first user to put a time limit on the swap request that allows the request to be active for a certain period of time. In this way, if the second user does not respond to the swap request in a timely manner, the first user can send a new swap request to a different provider (e.g., a third user) all without having to manually track down the second user.

Manual Scheduling by Selected Providers

In yet another embodiment, the system 100 may be configured to allow a first business (e.g., a first hospital) to create a shift schedule and select one or more providers to schedule themselves in open shifts. For example, a hospital may fill open shifts by selecting preferred providers and allowing them to assign themselves to open shifts. The system may further be configured to allow the selected providers to select shifts all at the same time, provide certain providers first dibs on selecting shifts before the other providers (e.g., senior providers get first option before more junior providers, higher rated providers select before lower rated providers, etc.).

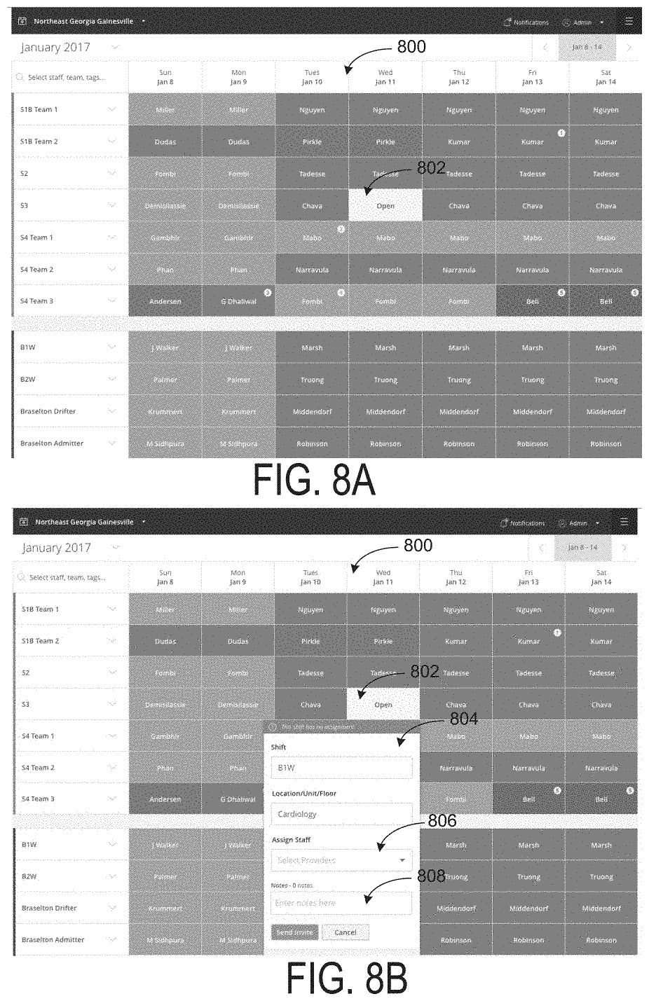

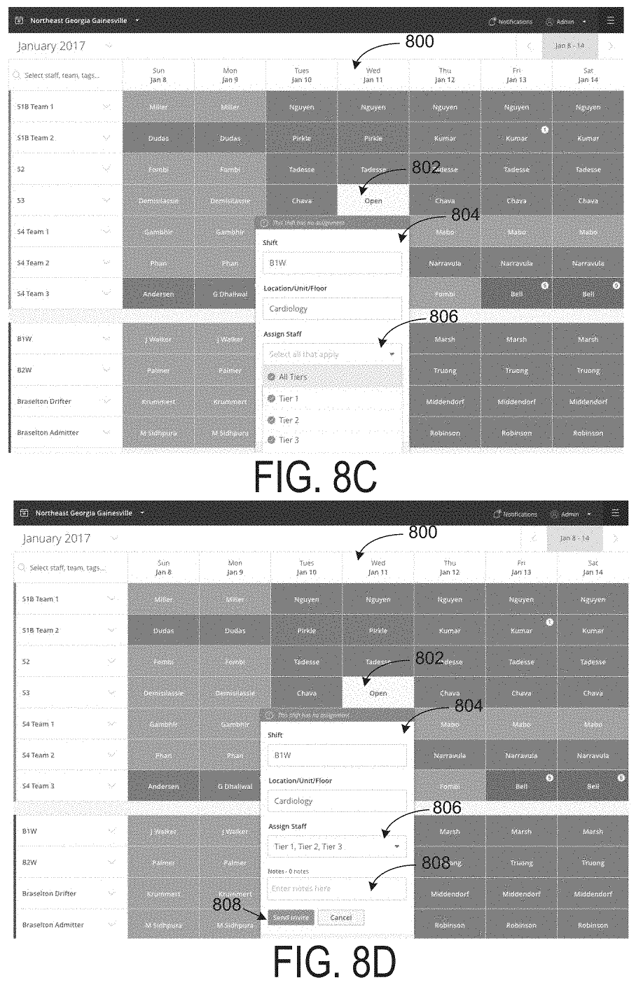

Referring to FIG. 8A, the scheduler for the first hospital has created a schedule 800 with an open shift 802. Referring to FIG. 8B, when the scheduler selects the open shift 802 on the schedule, an invite box 804 opens that allows the scheduler to select one or more users to fill the open shift in the schedule. The invite box 804 allows the schedule to select one or more users in a “select provider” drop down menu 806. Referring to FIG. 8C, the scheduler may pick one or more users by name or they may pick users based on groupings such as “all tiers”, “tier 1”, “tier 2”, “tier 3”, etc. Each tier can be based on one or more of a user's seniority level, a user's rating level, a compensation amount that the user has indicated that they want for working a shift, or any other suitable criteria that can be used to group providers. Additionally, and referring to FIG. 8D, the invite box 804 includes a notes field 808 where the scheduler may include the compensation amount available to a user that works the open shift. The notes field may also contain any other information that is associated with the open shift. Once the scheduler is finished entering information in the invite box 804, the schedule can select an “send invite” menu item 810 to send the invite to the one or more selected providers.

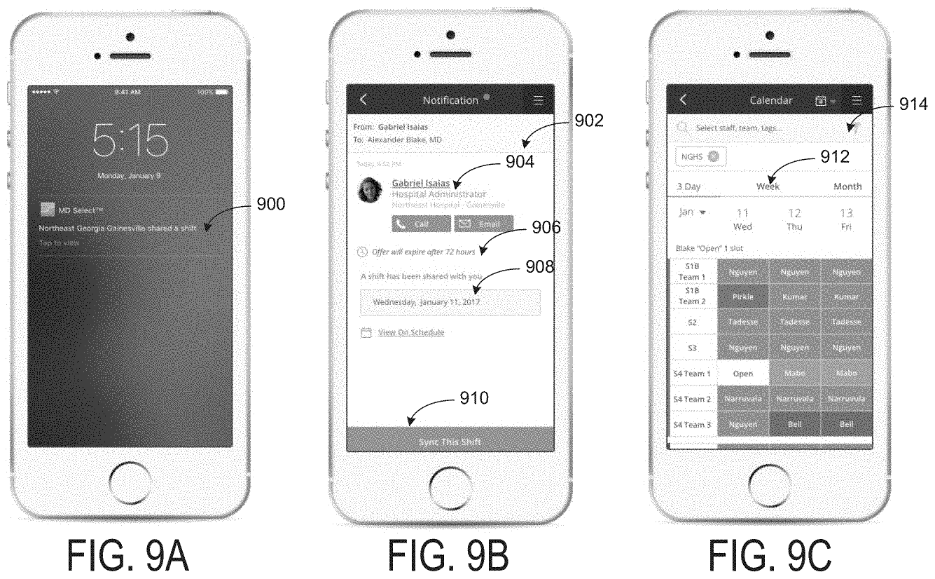

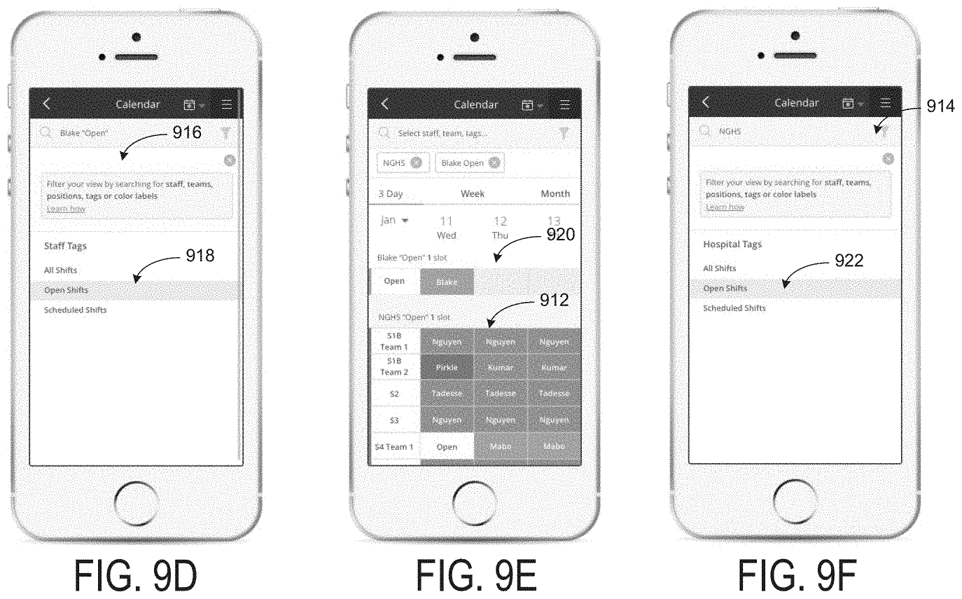

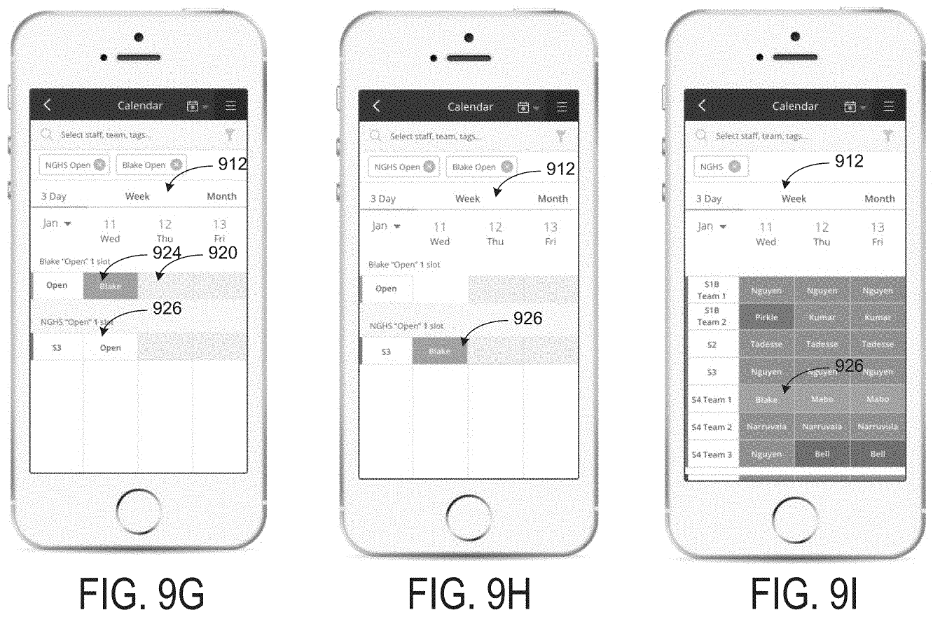

Continuing with the above example and referring to FIG. 9A, once the scheduler sends the invite, one of the selected providers will receive an electronic notification 900 on their computing device (e.g., a smart phone, a tablet, a laptop, etc.). When the user selects the notification 900, it brings the user to a user interface 902B for the system 100 as shown in FIG. 9B where the user can view the invite information. The invite may indicate the contact information 904 for the hospital administrator (e.g., scheduler, department manager, etc.), an offer time limit 906 (e.g., when the offer to work the open shift expires, and shift information 908. The user interface 902 may also provide a “sync this shift” menu item 910 that allows the system 100 to show the open shift in a calendar view 912 (FIG. 9C).

Referring to FIG. 9D, the user may filter the view in the schedule by selecting a “filter” menu item 914 (FIG. 9C) that opens a filter user interface 916. The user can then select a “open shifts” menu item 918 that overlays the user's open day 920 adjacent to and aligned with the calendar view 912. Thus, the user can easily compare their open days with the unassigned shifts available to them. Referring to FIG. 9F, the user can also select an “open shifts” menu item 922 using the filter 914 thereby simplifying the calendar view that is provided to the user, as shown in FIG. 9G. Thus, in the view shown in FIG. 9G, only the open shifts for the hospital are shown in aligned with the open dates for the user. In this example, the user may select open date 924 in FIG. 9G and drag it onto the unassigned shift (e.g., open shift) 926 for the hospital as shown in FIG. 9H. The system 100 may automatically update the schedule and present the updated calendar schedule view 912, as shown in FIG. 9I, with the user now assigned to the previously open shift 926 (FIG. 9G). In various embodiments, once a user assigns themselves to an open shift, the system updates the schedule and provides notice to other providers that may have been notified of the open shift. In this way, only one user can schedule themselves to the open shift.

Resource Marketplace

From the above description of the system and the use cases discussed herein, one of skill in the art should understand that the system 100 may be configured to create a marketplace for a geographic area (e.g., a city, a state, a region or the united states) for assigning resources (e.g., providers of services, people, equipment, etc.) to requesting parties. For example, in the case of health care workers, the system may allow health care providers from around the country to request work shifts from any organization that is seeking to fill unassigned work shifts. In addition, the system may also be configured to allow a user to request electronic credentialing at a particular facility (e.g., hospital, hospital group, facility, etc.) that is requesting a shift to be filled by providing information via the system 100 that is electronically shared with the facility. In various embodiments, the system 100 may automatically verify a health care providers license information, education, etc. through electronic state records and carry out the credentialing process on behalf of the facility. In this way, credentialing of health care providers is automated and streamlined eliminating the need to send papers through the mail and expediting the process so that healthcare providers can fill shifts on short notice. In other embodiments, the system may collect the necessary information from the healthcare provider, including uploaded support documentation, and allow the facility to verify the information. Once the facility credentials the healthcare provider, the system may update their electronic records and assign work shifts to the credentialed healthcare provider. In particular embodiments, the disclosed methods and systems may be applied to any other organizations, entities, and/or businesses that may have a need to assign resources of any type to requesting parties.

In other embodiments, the system may also be used as a resource marketplace for equipment. For example, business that is in need of specialized equipment may register with the system and upload request for equipment to the system 100. For example, if a business is in need of a specialized crane for a particular number of days at a particular location, the information can be loaded to the system and notification may be sent to equipment owners. The equipment owners may then evaluate the request, submit a bid including requested compensation for use of the equipment and the system can either automatically assign the owners bid to the request or allow the requester to manually assign the bid to the request. Once a bid is assigned to a request, the system would notify the bidder that the requester has accepted the bid.

### Determination of Potential Shift Candidates

By using the disclosed systems and methods, the system may, in various embodiments, determine one or more suitable candidates that are most likely to accept an offer to fill an unfilled shift. By offering shifts to candidates that are most likely to accept them, the system may, for example, increase the efficiency of resource utilization and reduce wasted candidate time (e.g., by not offering shifts to candidates who are unlikely to be interested in such shifts). If a particular shift remains unfilled despite being offered to candidates more likely to accept such a shift, the system may then offer the particular shift to one or more candidates that are less likely to accept the shift until the shift is filled or until all suitable candidates have been offered the particular shift.

In various embodiments, the system may be configured to identify potential candidates for filling shifts at one or more health care facilities. The system may also, or instead, be configured to prioritize and/or generate an ordered listing of such candidates based on various criteria. The system may base such prioritization and/or ordering on the determined likelihood of each particular candidate to accept a particular unfilled work shift. The system may obtain shift and/or candidate criteria from one or more data repositories. The system may also, or instead, determine shift and/or candidate criteria using data obtained from one or more data repositories. The system may also, or instead, collect or determine candidate criteria by prompting candidates for information indicating such criteria or that may be used to determine such criteria. For example, the system may present a questionnaire on a graphical user interface and prompt the user to provide respective answers (e.g., via the graphical user interface) to one or more questions in the questionnaire. In another example, the system may query a user for information using a graphical user interface and collect information from the user via the graphical user interface (or other suitable user interface). The system may use the information collected from a user as criteria used as described herein and/or to determine criteria used as described herein. The system may also, or instead, store such information collected from the user and/or determined based on information collected from the user in one or more data repositories for use in any exemplary embodiment described herein.

In particular embodiments, the criteria identified and/or determined for each prospective candidate for filling a particular unfilled work shift (“shift candidate”) can include, but is not limited to: (a) whether the particular candidate has worked the particular shift in the past; (b) the number of times in the past that particular candidate has worked the particular shift; (c) whether the particular candidate has worked the particular shift in the past as part of a shift swapping agreement; (d) the number of times in the past the particular candidate has worked the particular shift as part of a shift swapping agreement; (e) the number of times in the past the particular candidate has worked any shift as part of a shift swapping agreement generally; (f) whether the particular candidate has worked a shift in the past that occurs in a same timeframe but at a different day than the unfilled shift; and/or (g) whether the particular candidate has worked a shift in the past that occurs on a same day of the week but during a different timeframe than the unfilled shift.

In particular embodiments, the criteria identified and/or determined for each prospective shift candidate may be related to one or more respective characteristics of one or more other candidates and/or coworkers, such as an individual requesting a shift swap, and can include, but is not limited to: (a) whether the particular candidate has worked frequently with other particular individuals who are scheduled to work the particular unfilled shift; (b) whether the particular candidate has corresponded in the past with the individual requesting a shift swap (e.g., via the system); (c) whether the particular candidate has swapped shifts in the past with the individual requesting a shift swap; (d) whether the particular candidate is in a list of contacts of the individual requesting a shift swap (e.g., stored in the system); (e) whether the particular candidate has worked frequently in the past with the individual requesting a shift swap; and/or (f) whether the particular candidate also has a shift swap request pending that could be satisfied by the individual requesting a shift swap (e.g., as determined by the system and associated scheduling software).

In particular embodiments, the criteria identified and/or determined for each prospective shift candidate may be related to one or more measurements of the particular shift candidate's shift acceptance behavior, such as, but not limited to: (a) a rate of acceptance by the particular candidate of past requests to fill shifts generally; (b) a rate of acceptance by the particular candidate of past requests to fill shifts occurring in the same day as the request; (c) a rate of acceptance by the particular candidate of past requests to fill shifts in a particular timeframe; and/or (d) a rate of acceptance by the particular candidate of past requests to fill shifts on a particular day of the week.

In various embodiments, the system may assign a value (e.g., numerical value) to each criterion used to evaluate shift candidates and then use these numerical values to generate a score. The system may determine a score for a particular shift candidate that reflects the candidate's likelihood of acceptance of an offer to fill a particular shift based on the numerical values. The system may determine such a score by summing all the respective values of the various criteria used. In various embodiments, the system may apply a weighting factor to one or more of the criteria values for any suitable reason prior to summing all the values of the various criteria used. In other examples, the system may employ any other suitable algorithm or formula to determine a score for a particular shift candidate. The system may determine scores, for example, based on a likelihood of acceptance of a request to fill an unfilled shift for one or more candidates. The system may then rank the available candidates to determine an order in which to offer candidates an unfilled shift. In particular embodiments, the system may include a neural network comprising nodes that mimic the operation of the human brain, a training mechanism that analyzes supplied information (e.g., health care worker data, shift data, scheduling data, etc.), and a shift fulfillment engine for determining and ranking candidates for filling one or more shifts, for offering shifts to candidates, and for entering candidate information into a schedule when shifts are accepted by candidates. The neural network may execute any of the modules described herein (e.g., using a training mechanism and/or a shift fulfillment engine) to substantially automatically assign candidates to shifts, include the Shift Candidate Scoring Module 1000 and the Shift Fulfillment Module 1100.

### Shift Candidate Scoring Module

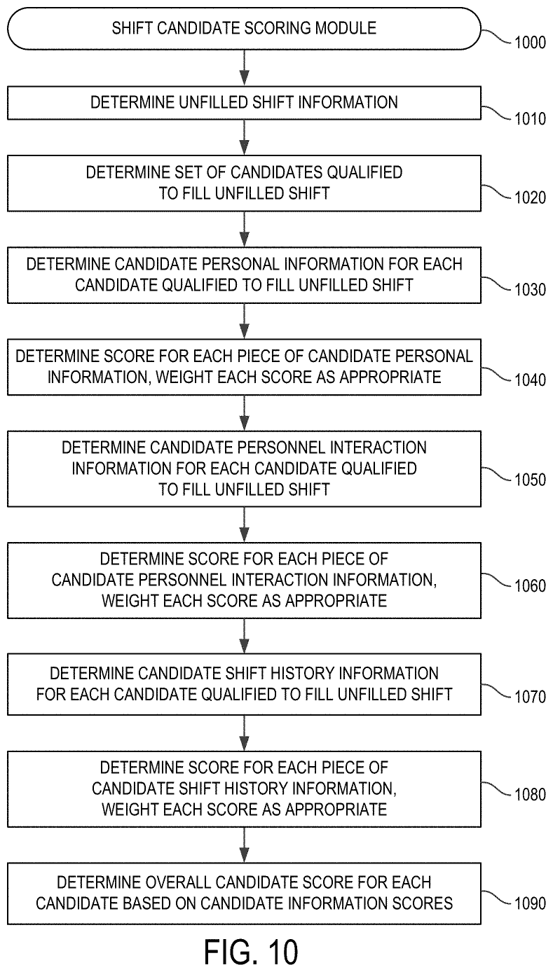

In various embodiments, the system may execute the Shift Candidate Scoring Module 1000 shown in FIG. 10 to determine respective scores for each of one or more candidates that may be available to fill an unfilled shift at a heath care facility. The system may use these respective scores to rank candidates in an order that the system may then use to offer the unfilled shifts to such candidates. The system begins, at Step 1010, by determining information related to a shift that needs to be filled by one or more healthcare workers (“unfilled shift”). The system may determine unfilled shift information using any means described herein or otherwise, for example by receiving some or all of such information from a user, by retrieving some or all of such information from a data repository, and/or by processing data received from a user and/or a data repository to generate some or all of such information. Unfilled shift information may include, but is not limited to: (a) the time, day, and/or date of the shift; (b) a recurrence of the shift, if any (e.g., the same day and time each week for a particular number of weeks); (c) one or more qualifications required for the shift; (d) the location of the shift (e.g., in examples where the system schedules shifts for multiple facilities); and/or (e) compensation for performing the shift; etc.

At Step 1020, the system may determine a set of candidates that are qualified to fill the unfilled shift. For example, the system may select, from among a pool of candidates (e.g., available at a data repository), a set of candidates that meet at least the minimum qualifications for filling the shift based on the education, experience, training, and/or other qualifications of such candidates. The system may also, or instead, use any other criteria to determine a set of candidates qualified to fill an unfilled shift.

At Step 1030, the system may, for each respective candidate in the set determined at Step 1020, determine personal information of the particular candidate. Such personal information may include, but is not limited to: (a) a home address and/or current location of the particular candidate; (b) a normal and/or preferred work location of the particular candidate; (c) one or more particular days of the week that the particular candidate is available to work a shift; (d) one or more particular sets of hours during which the particular candidate is available to work a shift; (e) whether the particular candidate has access to transportation (e.g., to travel to various facility locations); (f) a distance from the particular candidate's home that the particular candidate is willing to travel to work a shift; and/or (g) a distance from the particular candidate's normal work location that the particular candidate is willing to travel to work a shift.

At Step 1040, the system may determine a score for each piece of candidate personal information. In particular examples, a score may be a binary representation of a particular piece of information that indicates whether the respective candidate is likely to be available for the particular shift. For example, for a piece of candidate personal information that indicates the work hours that the particular candidate is available, the system may assign a ‘1’ when the candidate's available work hours include the hours of the unfilled shift and a ‘0’ when the candidate's available work hours do not include the hours of the unfilled shift. In particular examples, a score may have any of a variety values representing the particular piece of information. For example, for a piece of candidate personal information that indicates a home address or location of the particular candidate, the system may assign a score representing a number of miles from the home of the particular candidate to the healthcare facility at which the unfilled shift is to be performed. The system may use any suitable means and methods of determining or calculating a score for any piece of candidate personal information.

Further, at Step 1040, the system may apply a weight to a score for a particular piece of candidate personal information. Such weights may reflect certain characteristics or criteria associated with an unfilled shift. For example, for a shift that starts within a short period of time, the system may apply a non-zero weighting factor to a score for a piece of candidate personal information that indicates that the respective candidate lives close to the facility at which the unfilled shift is to be performed (e.g., by multiplying the weighting factor by the score), or otherwise decrease the score. In another example, for an unfilled shift that occurs outside the available work hours for a particular candidate, the system may apply a weighting factor that is less than zero to, or otherwise decrease, a score for a piece of candidate personal information that indicates that the respective candidate's work hours do not include those of the unfilled shift. The system may use any suitable means and methods of determining and/or applying a weight or weighting factor to any score for any piece of candidate personal information. The system may also, or instead, apply no weighting factor to any particular score for any piece of candidate personal information.

At Step 1050, the system may, for each candidate in the set determined at Step 1020, determine personnel interaction information for the particular candidate. Such personnel interaction information may include information related to one or more coworkers with whom the particular candidate has interacted, including, but not limited to: (a) one or more coworkers with whom the particular candidate has worked in the past and who are scheduled to work the unfilled shift; (b) a frequency with which the particular candidate has worked with one or more coworkers scheduled to work the unfilled shift; (c) one or more coworkers included in the contacts list of the particular candidate and who are scheduled to work the unfilled shift; (d) a frequency with which the particular candidate has worked with one or more coworkers scheduled to work the unfilled shift and who are also included in the contacts list of the particular candidate; (e) any specific positive, negative, or neutral interaction information (e.g., personal history, one or more peer ratings, one or more supervisor ratings, human resources feedback, incidents, etc.) associated with any combination of the particular candidate and any one or more coworkers scheduled to work the unfilled shift; (f) a frequency with which the particular candidate has communicated (e.g., via electronic communications) with one or more coworkers scheduled to work the unfilled shift; and/or (g) any other suitable personnel interaction information.

At Step 1060, the system may determine a score for each piece of candidate personnel interaction information. As with other scores described herein, such scores may each be a binary representation of a particular piece of information that indicates whether the respective candidate is likely to be available for the particular shift or may have any of a variety values representing the particular piece of information. For example, for a piece of candidate personnel interaction information that indicates whether the particular candidate has worked with any of the coworkers scheduled to work the unfilled shift, the system may assign a ‘1’ when the candidate has worked with such coworkers and a ‘0’ when the candidate has not worked with any of such coworkers. In another example, for a piece of candidate personnel interaction information that indicates ratings assigned to the particular candidate by one or more coworkers and/or supervisors scheduled to work the unfilled shift, the system may assign a score representing a sum of positive ratings minus a sum of negative ratings. The system may use any suitable means and methods of determining or calculating a score for any piece of candidate personnel interaction information.

Further at Step 1060, the system may apply a weight to a score for a particular piece of candidate personnel interaction information. As with other information described herein, such weights may reflect certain characteristics or criteria associated with an unfilled shift. For example, for a shift that is to be staffed by one or more coworkers with whom the particular candidate has worked frequently in the past, the system may apply a weighting factor that is greater than one to, or otherwise increase, a score for a piece of candidate personnel interaction information that indicates that the respective candidate has worked with personnel assigned to the unfilled shift. In another example, for an unfilled shift that is to be staffed by one or more coworkers with whom the particular candidate has had a negative personal interaction in the past (e.g., involved in a reported human resources incident, has requested not to be assigned to work together, etc.), the system may apply a weighting factor that is less than zero to, or otherwise decrease, a score for a piece of candidate personal information that indicates that the respective candidate has worked with personnel assigned to the unfilled shift. The system may use any suitable means and/or methods of determining or applying a weight or weighting factor to any score for any piece of candidate personnel interaction information. The system may also, or instead, apply no weighting factor to any particular score for any piece of candidate personnel interaction information.

At Step 1070, the system may, for each candidate in the set determined at Step 1020, determine shift history information for the particular candidate. Such shift history information may include information related to the particular candidate's history of working shifts in the past, including, but not limited to: (a) whether the particular candidate has worked the unfilled shift in the past; (b) if the particular candidate has worked the unfilled shift in the past, whether such work was in response to accepting an offered unfilled shift or part of a previous regular work schedule; (c) whether the particular candidate has worked a shift at the same time as, but on a different day than, the unfilled shift in the past; (d) whether the particular candidate has worked a shift on the same day as, but at a different time than, the unfilled shift in the past; (e) whether and/or how frequently the particular candidate has accepted offers of unfilled shifts in the past; (f) how quickly the candidate accepts offers to fill unfilled shifts (e.g., for unfilled shifts that occur relatively soon); (g) particular details of past accepted offers to fill unfilled shifts (e.g., candidate often accepts offered unfilled shifts occurring directly before or after the candidate's regularly schedule shift, candidate never accepts offered unfilled shifts occurring during the candidate normal day off, etc.); (h) whether the particular candidate has a currently pending request to swap a shift that may be satisfied by the offered unfilled shift; and/or (i) any other suitable candidate shift history information.

At Step 1080, the system may determine a score for each piece of candidate shift history information. As with other scores described herein, such scores may each be a binary representation of a particular piece of information that indicates whether the respective candidate is likely to be available for the particular shift or may have any of a variety values representing the particular piece of information. For example, for a piece of candidate shift history interaction information that indicates that whether the particular candidate often accepts offers to work unfilled shifts, the system may assign a ‘1’ if the candidate usually accepts such offers and a ‘0’ if the candidate rarely or never accepts such offers. In another example, for a piece of candidate shift history interaction information that indicates how likely a particular candidate is to accept offers to work unfilled shifts, the system may assign a score representing percentage of times that the particular candidate has accepted offers to work unfilled shifts. The system may use any suitable means and/or methods of determining and/or calculating a score for any piece of candidate personnel interaction information.

Further at Step 1080, the system may apply a weight to a score for a particular piece of candidate shift history information. As with other information described herein, such weights may reflect certain characteristics or criteria associated with an unfilled shift. For example, for an unfilled shift that is scheduled to occur the same day, the system may apply a weighting factor of greater than one to, or otherwise increase, a score for a piece of candidate shift history information that indicates that the respective candidate frequently accepts offers to fill shifts occurring in the same day that the offer was made. On the other hand, the system may apply a weighting factor of less than one, or otherwise decrease, a score for a piece of candidate shift history information that indicates that the respective candidate rarely or never accepts offers to fill shifts occurring in the same day that the offer was made. The system may use any suitable means and/or methods of determining or applying a weighting factor to any score for any piece of candidate shift history information. The system may also, or instead, apply no weighting factor to any particular score for any piece of candidate shift history information.

At Step 1090, the system may determine an overall shift acceptance likelihood score for each particular candidate based on the particular pieces of candidate information related to candidate personal information, candidate personnel interaction information, and candidate shift history information (e.g., such as that determined and scored at Steps 1030-1080). This score may, for example, be a sum of the scores (e.g., weighted scores, if applicable) determined for the particular pieces of candidate information or may be determined using any suitable formula or algorithm.

In particular examples, the system may generate and adjust an overall score for a particular candidate as each individual piece of candidate information (e.g., related to candidate personal information, candidate personnel interaction information, and/or candidate shift history information) is assessed. For example, where the overall score is a sum of the scores of each individual piece of candidate information, the system may maintain a running score, adding each score for each individual piece of candidate information to the running score as needed. The system may also apply weightings as suitable in this fashion, applying a weighting factor to each score for each individual piece of candidate information and then adding the weighted score for the individual piece of candidate information to the running score. The system may alternatively use any suitable means and/or methods of determining an overall score representing the likelihood that a particular candidate will accept an offer to fill an unfilled shift. The system may store any of the scores determined by the Shift Candidate Scoring Module in one or more data repositories, e.g., for future use and/or reference.

### Shift Fulfillment Module

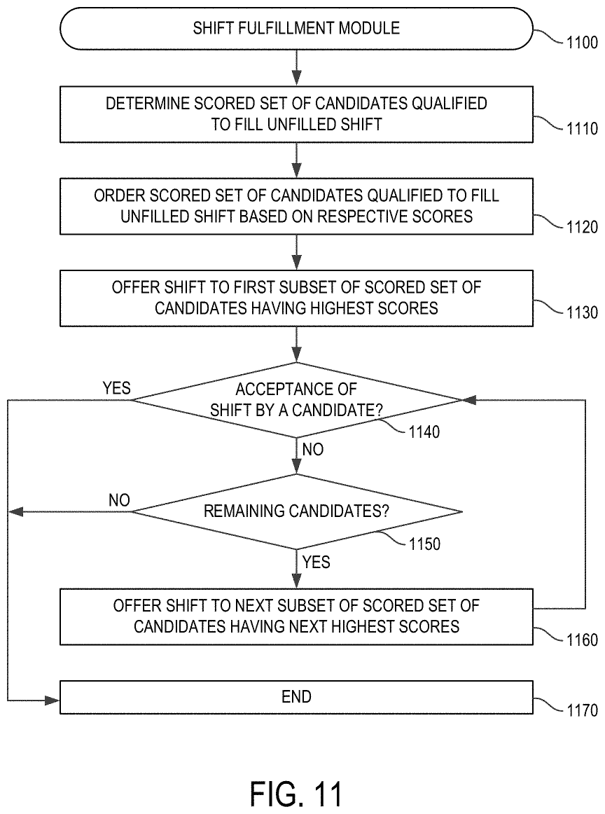

In various embodiments, the system may execute the Shift Fulfillment Module 1100 shown in FIG. 11 to fill a particular unfilled shift by one or more candidates for which shift acceptance likelihood scores have been determined for the particular unfilled shift (e.g., as described in regard to the Shift Candidate Scoring Module above). The system may use overall shift acceptance likelihood scores to rank candidates and then offer the unfilled shifts to such candidates in the order of ranking. The system begins at Step 1110, by determining the set of one or more candidate healthcare workers that have been scored and are qualified to fill a particular unfilled shift. The system may determine this set of candidates by accessing one or more data repositories and/or using any means described herein or otherwise. At Step 1120, the system may order the set of candidates based on the respective overall shift acceptance likelihood score for each candidate.

At Step 1130, the system may offer the particular unfilled shift to a first subset of the set of candidates having the highest overall shift acceptance likelihood scores. For example, the system may be configured to initially offer an unfilled shift to a specific number of highest scoring candidates (e.g., 5, 10, 20, etc.). In a particular example, the system may determine the top five highest scoring candidates. The system may then offer the unfilled shift to these top five highest scoring candidates first, before offering the unfilled shift to any other candidate if necessary. In a particular example, the system may be configured to offer an unfilled shift to the candidates who each have an overall shift acceptance likelihood score that is greater than a predefined threshold value. In another particular example, the system may be configured to offer an unfilled shift to the candidates who are each within a top predefined percentage (e.g., top 10%, 20%, 50%, etc.) of overall shift acceptance likelihood scores from among the overall shift acceptance likelihood scores for all candidates.

At Step 1140, the system may determine whether one of the candidates initially offered the unfilled shift has accepted the shift. If the shift has been accepted, the system may end the process of attempting to fill the unfilled shift and proceed to terminate the process at Step 1170. The system may also generate an indication to a user that the shift has been filled (e.g., by presenting a suitable indication on a graphical user interface).

If, at Step 1140, no candidate has accepted an offer to fill the unfilled shift (e.g., no acceptance received after a preconfigured period of time), the system may determine, at Step 1150, whether there are remaining candidates in the set of one or more candidates for which shift acceptance likelihood scores have been determined for the particular unfilled shift. If there are no remaining candidates to whom to offer the unfilled shift (or if any other suitable criteria are satisfied), the system may end the process of attempting to fill the unfilled shift and proceed to terminate the process at Step 1170. In this case, the system may also generate an indication to a user that the shift could not be filled (e.g., by presenting an indication on a graphical user interface).

If the system determines, at Step 1150, that there are remaining candidates in the set of one or more candidates for which shift acceptance likelihood scores have been determined for the particular unfilled shift, at Step 1160 the system may offer the unfilled shift to the subset of candidates having the next highest shift acceptance likelihood scores after the first subset with the highest shift acceptance likelihood scores (as determined at Step 1130). As with Step 1130, the system may be configured to offer the unfilled shift to a specific number of next-highest scoring one or more candidates (e.g., 5, 10, 20, etc.). In a particular example, the system may determine the next five highest scoring candidates after the top five highest scoring candidates. The system may then offer the unfilled shift to these next five highest scoring candidates first, before offering the unfilled shift to any other candidate if necessary. In a particular example, the system may be configured to offer an unfilled shift to the candidates who each have an overall shift acceptance likelihood score that is greater than a predefined threshold value, but less than the predefined threshold value used to determine the candidates to whom to make the first (or prior) offer to fill the unfilled shift. In another particular example, the system may be configured to offer the unfilled shift to the one or more candidates who are each within next-highest predefined percentage (e.g., second or subsequent 10%, 20%, 50%, etc.) of overall shift acceptance likelihood scores from among the overall shift acceptance likelihood scores for all candidates.

The system may then return to Step 1140 to determine if the offer to fill the unfilled shift has been accepted and, if not, whether there are additional candidates to whom to offer the shift. The system may iteratively perform Steps 1140-1160 until the unfilled shift is filled or until there are no more candidates to whom to offer the shift. In response to the shift being accepted, the system may notify a user that the shift has been filled (e.g., by presenting an indication on a graphical user interface). Alternatively, in response to the shift remaining unfilled after the set of candidates has been exhausted, the system may notify a user that the shift could not be filled (e.g., by presenting a suitable indication on a graphical user interface).

### CONCLUSION

Many modifications and other embodiments of the invention will come to mind to one skilled in the art to which this invention pertains having the benefit of the teachings presented in the foregoing descriptions and the associated drawings. While examples discussed above cover the use of the invention in the context of scheduling work shifts in healthcare facilities, various embodiments may be used in any other suitable context (e.g., within the context of scheduling other types of work shifts, or within the context of scheduling other types of activities such as volunteers at a shelter, parents assigned tasks related to kids athletic programs, resources assigned in a school setting, etc.). Also, it should be understood that the system may be adapted to allow staff personnel (e.g., staff physicians, advanced practitioners, and/or nurses to sign up to work extra shifts for additional compensation beyond their salary (e.g., overtime). For example, the system may allow staff personnel to select to work extra shifts (e.g., along with independent-contractor physicians, advanced practitioners, and nurses) after the system has scheduled all available staff personnel to work full schedules. Therefore, it is to be understood that the invention is not to be limited to the specific embodiments disclosed and that modifications and other embodiments are intended to be included within the scope of the appended claims. Although specific terms are employed herein, they are used in a generic and descriptive sense only and not for the purposes of limitation.

What is claimed is:

1.-2. (canceled)

3. A computer architecture comprising:
at least one primary computer processor communicatively connected to a primary computer memory;
wherein the primary computer memory stores computer-executable instructions that, when executed by the at least one primary computer processor, are operable for managing a healthcare provider scheduling system by: (1) automatically identifying a particular candidate medical provider to provide medical services within a particular time slot at a particular healthcare facility, and (2) automatically extending an offer to the particular candidate medical provider to provide medical services within the particular time slot at the particular healthcare facility by:
receiving, by the at least one primary computer processor, timeslot criteria associated with a plurality of unfilled timeslots, the plurality of unfilled timeslots comprising a first unfilled timeslot at the particular healthcare facility and a second unfilled timeslot;
receiving, by the at least one primary computer processor, qualification data for each of a plurality of candidate healthcare providers;
receiving, by the at least one primary computer processor, personal data for each of the plurality of candidate healthcare providers, the personal data comprising at least one type of data selected from a group consisting of:
(a) a respective home location of the candidate healthcare provider;
(b) a respective normal work location of the candidate healthcare provider; and
(c) one or more respective days of the week on which the candidate healthcare provider is available to work;
receiving, by the at least one primary computer processor, interaction data for each of the plurality of candidate healthcare providers, the interaction data comprising at least one type of data selected from a group consisting of:
(a) one or more individuals with whom the candidate healthcare provider has worked in the past and who is scheduled to work at the particular healthcare facility in the first unfilled timeslot; and
(b) one or more individuals who is scheduled to work at the particular healthcare facility in the first unfilled timeslot and who is in a contact list associated with the candidate healthcare provider;
receiving, by the at least one primary computer processor, historical operation data for each of the plurality of candidate healthcare providers, the historical operation data comprising at least one type of data selected from a group consisting of:
(a) an indication of a frequency that the candidate healthcare provider has accepted past offers to fill unfilled timeslots;
(b) an indication of the candidate healthcare provider's response time to past offers to fill unfilled timeslots;
(c) an indication of whether the candidate healthcare provider has accepted a past offer to fill an unfilled timeslot that occurred in a same time period as the first unfilled timeslot; and
(d) an indication of whether the candidate healthcare provider has accepted a past offer to fill an unfilled timeslot that occurred on a same day as the first unfilled timeslot;
automatically identifying, by the at least one primary computer processor, based at least in part on the qualification data and the timeslot criteria, one or more of the candidate healthcare providers that are qualified to fill the first unfilled timeslot;
for each particular one of the identified qualified candidate healthcare providers:
automatically analyzing, by the at least one primary computer processor, the respective personal data of the particular identified qualified candidate healthcare provider to determine a respective personal data score for the particular identified qualified candidate healthcare provider;
automatically analyzing, by the at least one primary computer processor, the respective interaction data of the particular identified qualified candidate healthcare provider to determine a respective interaction score for the particular identified qualified candidate healthcare provider; and
automatically analyzing, by the at least one primary computer processor, the respective historical operation data of the particular identified qualified candidate healthcare provider to determine a respective historical operation score for the particular identified qualified candidate healthcare provider;
using machine learning techniques to automatically determine, by the at least one primary computer processor, a respective overall timeslot acceptance likelihood score for each particular identified qualified candidate healthcare provider based on the respective personal data score, the respective interaction score, and the respective historical operation score for the particular identified qualified candidate healthcare provider;
automatically ranking, by the at least one primary computer processor, the respective identified qualified candidate healthcare providers using the respective overall timeslot acceptance likelihood scores;
automatically identifying, by the at least one primary computer processor, a subset of highest-ranked identified qualified candidate healthcare providers; and
after identifying the subset of highest-ranked identified qualified candidate healthcare providers, for each identified qualified candidate healthcare provider within the identified subset of highest-ranked identified qualified candidate healthcare providers, automatically extending an offer to the identified qualified candidate healthcare provider by generating, by the at least one primary computer processor, a graphical user interface by:
configuring a first user-selectable timeslot item comprising an indication of the first unfilled timeslot and an indication of an offer to occupy the first unfilled timeslot on the graphical user interface; and
excluding a second user-selectable timeslot item comprising an indication of the second unfilled timeslot from the graphical user interface;
transmitting, by the at least one primary computer processor, a first instruction to a first application executing on a user device associated with the identified qualified candidate healthcare provider causing the user device to present the graphical user interface on the user device; and
in response to detecting a selection of the first user-selectable timeslot item, transmitting, by the at least one primary computer processor, a second instruction to a second application executing on a remote computing device causing the remote computing device to present an indication of acceptance of the offer to occupy the first unfilled timeslot by the identified qualified candidate healthcare provider.

4. The computer architecture of claim 3, wherein the computer-executable instructions further comprise instructions that, when executed by the at least one primary computer processor, are operable for:
determining, by the at least one primary computer processor, that no acceptance of the offer extended to each identified qualified candidate healthcare provider within the identified subset of highest-ranked identified qualified candidate healthcare providers has been received;
at least partially in response to determining that no acceptance of the offer extended to each identified qualified candidate healthcare provider within the identified subset of highest-ranked identified qualified candidate healthcare providers has been received, automatically identifying, by the at least one primary computer processor, a subset of next-highest ranked identified qualified candidate healthcare providers, wherein each identified qualified candidate healthcare provider within the identified subset of next-highest ranked identified qualified candidate healthcare providers has a respective overall timeslot acceptance likelihood score less than any overall timeslot acceptance likelihood score of any qualified candidate healthcare provider within the identified subset of highest-ranked identified qualified candidate healthcare providers; and
after identifying the subset of next highest-ranked identified qualified candidate healthcare providers, for each identified qualified candidate healthcare provider within the identified subset of next highest-ranked identified qualified candidate healthcare providers, automatically extending an offer to the identified qualified candidate healthcare provider by generating, by the at least one primary computer processor, a second graphical user interface comprising a second indication of the particular unfilled timeslot and a second indication of the offer to occupy the particular unfilled timeslot.

5. The computer architecture of claim 4, wherein the computer-executable instructions further comprise instructions that, when executed by the at least one primary computer processor, are operable for:
determining, by the at least one primary computer processor, that no acceptance of the second offer extended to each identified qualified candidate healthcare provider within the identified subset of next highest-ranked identified qualified candidate healthcare providers has been received;
at least partially in response to determining that no acceptance of the offer extended to each identified qualified candidate healthcare provider within the identified subset of next highest-ranked identified qualified candidate healthcare providers has been received, determining, by the at least one primary computer processor, that there are no identified qualified candidate healthcare providers that have not been extended one of the offer or the second offer; and
at least partially in response to determining that there are no identified qualified candidate healthcare providers that have not been presented with one of the offer or the second offer, generating, by the at least one primary computer processor, a third graphical user interface comprising an indication to a user that the particular unfilled timeslot remains unfilled.

6. The computer architecture claim 4, wherein the computer-executable instructions further comprise instructions that, when executed by the at least one primary computer processor, are operable for:
receiving, by the at least one primary computer processor, an acceptance of the second offer; and
at least partially in response to receiving the acceptance of the second offer, generating, by the at least one primary computer processor, a third graphical user interface comprising an indication to a user that the particular unfilled timeslot is filled.

7. The computer architecture of claim 3, wherein the personal data for each of the plurality of candidate healthcare providers further comprises one or more respective sets of hours during which the candidate healthcare provider is available to work.

8. The computer architecture of claim 3, wherein the interaction data for each of the plurality of candidate healthcare providers further comprises at least one type of data selected from a group consisting of:
(a) one or more peer ratings of the candidate healthcare provider; and
(b) one or more supervisor ratings of the candidate healthcare provider.

9. The computer architecture of claim 3, wherein:
the historical operation data for each of the plurality of candidate healthcare providers comprises the indication of the frequency that the candidate healthcare provider has accepted past offers to fill unfilled timeslots; and
the frequency that the candidate healthcare provider has accepted the past offers to fill unfilled timeslots comprises a percentage of the past offers to fill unfilled timeslots that the candidate healthcare provider has accepted.

10. The computer architecture of claim 3, wherein using machine learning techniques to automatically determine the respective overall timeslot acceptance likelihood score for each particular identified qualified candidate healthcare provider comprises applying a weighting factor to at least one piece of personal data for each particular identified qualified candidate healthcare provider.

11. The computer architecture of claim 3, wherein using machine learning techniques to automatically determine the respective overall timeslot acceptance likelihood score for each particular identified qualified candidate healthcare provider comprises applying a weighting factor to at least one piece of interaction data for each particular identified qualified candidate healthcare provider.

12. The computer architecture of claim 3, wherein using machine learning techniques to automatically determine the respective overall timeslot acceptance likelihood score for each particular identified qualified candidate healthcare provider comprises applying a weighting factor to at least one piece of historical operation data for each particular identified qualified candidate healthcare provider.

13. A computer system for generating offers to fill unassigned shifts at a healthcare facility, the system comprising:
at least one computer processor; and
computer memory storing computer-executable instructions that, when executed by the at least one computer processor, are operable for managing a healthcare provider scheduling system by: (1) automatically identifying a particular candidate medical provider to provide medical services within a particular time slot at a particular healthcare facility, and (2) automatically extending an offer to the particular candidate medical provider to provide medical services within the particular time slot at the particular healthcare facility by:
receiving timeslot criteria associated with a plurality of unfilled timeslots, the plurality of unfilled timeslots comprising a first unfilled timeslot at the particular healthcare facility and a second unfilled timeslot;
receiving qualification data for each of a plurality of candidate healthcare providers;
receiving personal data for each of the plurality of candidate healthcare providers, the personal data comprising at least one type of data selected from a group consisting of:
(a) a respective home location of the candidate healthcare provider;
(b) a respective normal work location of the candidate healthcare provider; and
(c) one or more respective days of the week on which the candidate healthcare provider is available to work;
receiving interaction data for each of the plurality of candidate healthcare providers, the interaction data comprising at least one type of data selected from a group consisting of:
(a) one or more individuals with whom the candidate healthcare provider has worked in the past and who is scheduled to work at the particular healthcare facility in the first unfilled timeslot; and
(b) one or more individuals who is scheduled to work at the particular healthcare facility in the first unfilled timeslot and who is in a contact list associated with the candidate healthcare provider;
receiving historical operation data for each of the plurality of candidate healthcare providers, the historical operation data comprising at least one type of data selected from a group consisting of:
(a) an indication of a frequency that the candidate healthcare provider has accepted past offers to fill unfilled timeslots;
(b) an indication of the candidate healthcare provider's response time to past offers to fill unfilled timeslots;
(c) an indication of whether the candidate healthcare provider has accepted a past offer to fill an unfilled timeslot that occurred in a same time period as the first unfilled timeslot; and
(d) an indication of whether the candidate healthcare provider has accepted a past offer to fill an unfilled timeslot that occurred on a same day as the first unfilled timeslot;
automatically identifying, based at least in part on the qualification data and the timeslot criteria, one or more of the candidate healthcare providers that are qualified to fill the first unfilled timeslot;
for each particular one of the identified qualified candidate healthcare providers:
automatically analyzing the respective personal data of the particular identified qualified candidate healthcare provider to determine a respective personal data score for the particular identified qualified candidate healthcare provider;
automatically analyzing the respective interaction data of the particular identified qualified candidate healthcare provider to determine a respective interaction score for the particular identified qualified candidate healthcare provider;
automatically analyzing the respective historical operation data of the particular identified qualified candidate healthcare provider to determine a respective historical operation score for the particular identified qualified candidate healthcare provider;
using machine learning techniques, automatically determining a respective overall timeslot acceptance likelihood score for each particular identified qualified candidate healthcare provider based on the respective personal data score, the respective interaction score, and the respective historical operation score for the particular identified qualified candidate healthcare provider;
automatically ranking the respective identified qualified candidate healthcare providers using the respective overall timeslot acceptance likelihood scores;
automatically identifying a subset of highest-ranked identified qualified candidate healthcare providers;
after identifying the subset of highest-ranked identified qualified candidate healthcare providers, for each identified qualified candidate healthcare provider within the identified subset of highest-ranked identified qualified candidate healthcare providers, automatically extending an offer to the identified qualified candidate healthcare provider by generating a graphical user interface by:
configuring a first user-selectable timeslot item comprising an indication of the first unfilled timeslot and an indication of an offer to occupy the first unfilled timeslot on the graphical user interface; and
excluding a second user-selectable timeslot item comprising an indication of the second unfilled timeslot from the graphical user interface;
transmitting, by the at least one primary computer processor, a first instruction to a first application executing on a user device associated with the identified qualified candidate healthcare provider causing the user device to present the graphical user interface on the user device; and
in response to detecting a selection of the first user-selectable timeslot item, transmitting, by the at least one primary computer processor, a second instruction to a second application executing on a remote computing device causing the remote computing device to present an indication of acceptance of the offer to occupy the first unfilled timeslot by the identified qualified candidate healthcare provider.

14. The computer system of claim 13, wherein determining the respective overall timeslot acceptance likelihood score for each particular identified qualified candidate healthcare provider based on the respective personal data score, the respective interaction score, and the respective historical operation score for each particular identified qualified candidate healthcare provider comprises:
applying a weighting factor to at least one piece of personal data for each particular identified qualified candidate healthcare provider;
applying a weighting factor to at least one piece of interaction data for each particular identified qualified candidate healthcare provider; and
applying a weighting factor to at least one piece of historical operation data for each particular identified qualified candidate healthcare provider.

15. The computer system of claim 13, wherein the personal data for each particular identified qualified candidate healthcare provider comprises one or more pieces of information selected from the group consisting of:
(a) whether the respective particular identified qualified candidate healthcare provider has access to transportation;
(b) a distance between a home location of the respective particular identified qualified candidate healthcare provider and a location of the particular healthcare facility; and
(c) a distance between a normal work location of the respective particular identified qualified candidate healthcare provider and the location of the particular healthcare facility; and
(d) an acceptable distance from the home location of the respective particular identified qualified candidate healthcare provider that the respective particular identified qualified candidate healthcare provider is willing to work.

16. The computer system of claim 13, wherein the interaction data for each particular identified qualified candidate healthcare provider comprises one or more pieces of information selected from the group consisting of:
(a) a frequency with which the respective particular identified qualified candidate healthcare provider has worked with one or more coworkers scheduled to work the particular unfilled timeslot;
(b) a frequency with which the respective particular identified qualified candidate healthcare provider has communicated with one or more coworkers scheduled to work the particular unfilled timeslot;
(c) a request by the respective particular identified qualified candidate healthcare provider to not work with one or more coworkers scheduled to work the particular unfilled timeslot; and
(d) a request by the respective particular identified qualified candidate healthcare provider to work with one or more coworkers scheduled to work the particular unfilled timeslot.

17. The computer system of claim 13, wherein the historical operation data for each particular identified qualified candidate healthcare provider comprises one or more pieces of information selected from the group consisting of:
(a) whether the particular unfilled timeslot occurs immediately before a normally scheduled timeslot for the respective particular identified qualified candidate healthcare provider;
(b) whether the particular unfilled timeslot occurs immediately after a normally scheduled timeslot for the respective particular identified qualified candidate healthcare provider;
(c) whether the respective particular identified qualified candidate healthcare provider has a pending request to swap a scheduled timeslot; and
(d) whether the respective particular identified qualified candidate healthcare provider has accepted a past offer to fill an unfilled timeslot that occurred on a same day as the particular unfilled timeslot.

18. A non-transitory computer-readable medium configured for storing computer-executable instructions for generating offers to fill unassigned shifts at a healthcare facility, the computer-executable instructions comprising instructions for managing a healthcare provider scheduling system by: (1) automatically identifying a particular candidate medical provider to provide medical services within a particular time slot at a particular healthcare facility, and (2) automatically extending an offer to the particular candidate medical provider to provide medical services within the particular time slot at the particular healthcare facility by:
receiving personal data for each of the plurality of candidate healthcare providers, the personal data comprising at least one type of data selected from a group consisting of:
(a) a respective home location of the candidate healthcare provider;
(b) a respective normal work location of the candidate healthcare provider; and
(c) one or more respective days of the week on which the candidate healthcare provider is available to work;
receiving interaction data for each of the plurality of candidate healthcare providers, the interaction data comprising at least one type of data selected from a group consisting of:
(a) one or more individuals with whom the candidate healthcare provider has worked in the past and who is scheduled to work at the particular healthcare facility in a first unfilled timeslot; and
(b) one or more individuals who is scheduled to work at the particular healthcare facility in the first unfilled timeslot and who is in a contact list associated with the candidate healthcare provider;
receiving historical operation data for each of the plurality of candidate healthcare providers, the historical operation data comprising at least one type of data selected from a group consisting of:
(a) an indication of a frequency that the candidate healthcare provider has accepted past offers to fill unfilled timeslots;
(b) an indication of the candidate healthcare provider's response time to past offers to fill unfilled timeslots;
(c) an indication of whether the candidate healthcare provider has accepted a past offer to fill an unfilled timeslot that occurred in a same time period as the first unfilled timeslot; and
(d) an indication of whether the candidate healthcare provider has accepted a past offer to fill an unfilled timeslot that occurred on a same day as the first unfilled timeslot;
automatically identifying, based at least in part on the qualification data and the timeslot criteria, one or more of the candidate healthcare providers that are qualified to fill the first unfilled timeslot;
for each particular one of the identified qualified candidate healthcare providers:
automatically analyzing the respective personal data of the particular identified qualified candidate healthcare provider to determine a respective personal data score for the particular identified qualified candidate healthcare provider;
automatically analyzing the respective interaction data of the particular identified qualified candidate healthcare provider to determine a respective interaction score for the particular identified qualified candidate healthcare provider; and
automatically analyzing the respective historical operation data of the particular identified qualified candidate healthcare provider to determine a respective historical operation score for the particular identified qualified candidate healthcare provider;
using machine learning techniques, automatically determining a respective overall timeslot acceptance likelihood score for each particular identified qualified candidate healthcare provider based on the respective personal data score, the respective interaction score, and the respective historical operation score for the particular identified qualified candidate healthcare provider;
automatically identifying, by the at least one primary computer processor, a subset of highest-ranked identified qualified candidate healthcare providers; and
after identifying the subset of highest-ranked identified qualified candidate healthcare providers, for each identified qualified candidate healthcare provider within the identified subset of highest-ranked identified qualified candidate healthcare providers, automatically extending an offer to the identified qualified candidate healthcare provider by generating, by the at least one primary computer processor, a graphical user interface by:
configuring a first user-selectable timeslot item comprising an indication of the first unfilled timeslot and an indication of an offer to occupy the first unfilled timeslot on the graphical user interface; and
excluding a second user-selectable timeslot item comprising an indication of a second unfilled timeslot from the graphical user interface;
transmitting, by the at least one primary computer processor, a first instruction to a first application executing on a user device associated with the identified qualified candidate healthcare provider causing the user device to present the graphical user interface on the user device; and
in response to detecting a selection of the first user-selectable timeslot item, transmitting, by the at least one primary computer processor, a second instruction to a second application executing on a remote computing device causing the remote computing device to present an indication of acceptance of the offer to occupy the first unfilled timeslot by the identified qualified candidate healthcare provider.

19. The non-transitory computer-readable medium of claim 18, wherein the offer to occupy the particular unfilled timeslot comprises an offer to work a plurality of recurring timeslots that repetitively occur on a same day of the week for a predetermined number of weeks.

20. The non-transitory computer-readable medium of claim 18, wherein the computer-executable instructions further comprise instructions for:
receiving an acceptance of the offer to occupy the particular unfilled timeslot; and
at least partially in response to receiving the acceptance of the offer to occupy the particular unfilled timeslot, generating an indication of the acceptance on the graphical user interface.

21. The non-transitory computer-readable medium of claim 18, wherein the computer-executable instructions further comprise instructions for:
receiving no acceptance of the offer to occupy the particular unfilled timeslot;
at least partially in response to receiving no acceptance of the offer to occupy the particular unfilled timeslot, determining that there are no identified qualified candidate healthcare providers that have not been extended the offer to occupy the particular unfilled timeslot; and
at least partially in response to determining that there are no identified qualified candidate healthcare providers that have not been extended the offer to occupy the particular unfilled timeslot, generating an indication that the particular unfilled timeslot remains unfilled on a second graphical user interface.

22. The non-transitory computer-readable medium of claim 18, wherein the computer-executable instructions further comprise instructions for:
determining, based on the timeslot criteria associated with the particular unfilled timeslot, healthcare provider qualifications for the particular unfilled timeslot; and
selecting, from a data repository, the plurality of candidate healthcare providers based on the healthcare provider qualifications for the particular unfilled timeslot.

23. A computer-implemented method comprising:
managing a healthcare provider scheduling system for: (1) automatically identifying a particular candidate medical provider to provide medical services within a particular time slot at a particular healthcare facility, and (2) automatically extending an offer to the particular candidate medical provider to provide medical services within the particular time slot at the particular healthcare facility by:
receiving, by one or more computer processors, timeslot criteria associated with a plurality of unfilled timeslots, the plurality of unfilled timeslots comprising a first unfilled timeslot at the particular healthcare facility and a second unfilled timeslot;
receiving, by one or more computer processors, qualification data for each of a plurality of candidate healthcare providers;
receiving, by one or more computer processors, personal data for each of the plurality of candidate healthcare providers, the personal data comprising at least one type of data selected from a group consisting of:
(a) a respective home location of the candidate healthcare provider;
(b) a respective normal work location of the candidate healthcare provider; and
(c) one or more respective days of the week on which the candidate healthcare provider is available to work;
receiving, by one or more computer processors, interaction data for each of the plurality of candidate healthcare providers, the interaction data comprising at least one type of data selected from a group consisting of:
(a) one or more individuals with whom the candidate healthcare provider has worked in the past and who is scheduled to work at the particular healthcare facility in the first unfilled timeslot; and
(b) one or more individuals who is scheduled to work at the particular healthcare facility in the first unfilled timeslot and who is in a contact list associated with the candidate healthcare provider;
receiving, by one or more computer processors, historical operation data for each of the plurality of candidate healthcare providers, the operation data comprising at least one type of data selected from a group consisting of:
(a) an indication of a frequency that the candidate healthcare provider has accepted past offers to fill unfilled timeslots;
(b) an indication of the candidate healthcare provider's response time to past offers to fill unfilled timeslots;
(c) an indication of whether the candidate healthcare provider has accepted a past offer to fill an unfilled timeslot that occurred in a same time period as the first unfilled timeslot; and
(d) an indication of whether the candidate healthcare provider has accepted a past offer to fill an unfilled timeslot that occurred on a same day as the first unfilled timeslot;
automatically identifying, based on the qualification data and the timeslot criteria, one or more of the candidate healthcare providers that are qualified to fill the first unfilled timeslot;
for each particular one of the identified qualified candidate healthcare providers:
automatically analyzing the respective personal data of the particular identified qualified candidate healthcare provider to determine a respective personal data score for the particular identified qualified candidate healthcare provider;
automatically analyzing the respective interaction data of the particular identified qualified candidate healthcare provider to determine a respective interaction score for the particular identified qualified candidate healthcare provider;
automatically analyzing the respective historical operation data of the particular identified qualified candidate healthcare provider to determine a respective historical operation score for the particular identified qualified candidate healthcare provider;
using machine learning techniques to automatically determine a respective overall timeslot acceptance likelihood score for each particular identified qualified candidate healthcare provider based on the respective personal data score, respective interaction score, and respective historical operation score for the particular identified qualified candidate healthcare provider;
using the respective overall timeslot acceptance likelihood scores to automatically rank the respective identified qualified candidate healthcare providers;
automatically identifying, based on the automatic ranking, a subset of highest-ranked identified qualified candidate healthcare providers; and
after identifying the subset, for each identified qualified candidate healthcare provider within the identified subset, automatically extending an offer to the identified qualified candidate healthcare provider by generating a graphical user interface by:
configuring a first user-selectable timeslot item comprising an indication of the first unfilled timeslot and an indication of an offer to occupy the first unfilled timeslot on the graphical user interface; and
excluding a second user-selectable timeslot item comprising an indication of the second unfilled timeslot from the graphical user interface;
transmitting, by the at least one primary computer processor, a first instruction to a first application executing on a user device associated with the identified qualified candidate healthcare provider causing the user device to present the graphical user interface on the user device; and
in response to detecting a selection of the first user-selectable timeslot item, transmitting, by the at least one primary computer processor, a second instruction to a second application executing on a remote computing device causing the remote computing device to present an indication of acceptance of the offer to occupy the first unfilled timeslot by the identified qualified candidate healthcare provider.
